SEQUENCE LISTING (12 sequences)

SEQ ID NO: 1 - DNA, 1110 bp, Bacillus licheniformis
atgagaaagcgctatgatacgatcgtgatcggcggaggcatcatcgggacttccatcgcttatcatcttgcgaaagcggggaaaaaaacggccgtgtttgaaagcggggaagtcggcaaaaaagcgacgagcgcggctgcgggcatgctcggcgcccatgcggaatgcgataaacccggtacctttttcgaatttgccagagccagtcaaaaagcctataagcgcctgacgggggaattgaaagacataagcggcattgatatcagaaggcatgacggagggatattaaagctggcattttcagaaagcgaccgcgaacacctgatgcaaatgggtgcactggattctgtggaatggcttgaagctgatgaagtatataaattagaaccgaatgccggaaaaggaattctcggcgccaattttatcagggacgacgtccatgtcgaaccggctgcagtgtgcagggcatttgccaggggagcccggatgctcggtgcagatgtcttcgagtatacgcccgttttatcgatagaatccgaagcgggggccgtccgcgtcacgtccgcttccggcacagcggaggcggagcacgctgtcattgcaagcggcgtgtggagcggagctttgtttaaacaaatcggcttagacaaaaggttttaccccgtcaaaggggagtgcctgtccgtatggaatgacggcatctctttgacgagaacgctttaccatgatcattgctacatcgttccgcgccatagcggaaggcttgtcgtcggcgcgacgatgaagcccggggactggaatgagcagccggaacttggcggaatcgaggagttgatccgaaaagccaagtcgatgctccctgggattgaatccatgaaaatcgaccaatgctgggccgggttgcgtcccgaaacaggggatggcaatccgtatatcggaagacatcctgaaaatgaccggattttgtttgctgccggacatttcagaaacggcatcctcctggcacctgcaaccggtgagatgatggcagacatgatcctcggcaatccggtcaaaacagagtggatcgaggcgtttaaagcggagcgaaaggaggctgtgcacaggtga

SEQ ID NO: 2 - protein, 369 aa, Bacillus licheniformis
MRKRYDTIVIGGGIIGTSIAYHLAKAGKKTAVFESGEVGKKATSAAAGMLGAHAECDKPGTFFEFARASQKAYKRLTGELKDISGIDIRRHDGGILKLAFSESDREHLMQMGALDSVEWLEADEVYKLEPNAGKGILGANFIRDDVHVEPAAVCRAFARGARMLGADVFEYTPVLSIESEAGAVRVTSASGTAEAEHAVIASGVWSGALFKQIGLDKRFYPVKGECLSVWNDGISLTRTLYHDHCYIVPRHSGRLVVGATMKPGDWNEQPELGGIEELIRKAKSMLPGIESMKIDQCWAGLRPETGDGNPYIGRHPENDRILFAAGHFRNGILLAPATGEMMADMILGNPVKTEWIEAFKAERKEAVHR

SEQ ID NO: 3 - DNA, 1110 bp, Bacillus licheniformis; identical to SEQ ID NO: 1

SEQ ID NO: 4 - protein, 369 aa, Bacillus licheniformis
MRKRYDTIVIGGGIIGTSIAYHLAKAGKKTAVFESGEVGKKATSAAAGMLSAHRECDKPGTFFEFARASQKAYKRLTGELRDISGIDIRRHDGGILKLAFSESDREHLMQMGALDSVEWLEADEVYKLEPNAGKGILGANFIRDDVHVEPAAVCRAFARGARMLGADVFEYTPVLSIESEAGAVRVTSASGTAEAEHAVIACGVWSGALFKQIGLDKRFYPVKGECLSVWNDGISLTRTLYHDHCYIVPRHSGRLVVGATMKPGDWNEQPELGGIEELIRKAKSMLPGIESMKIDQCWAGLRPETGDGNPYIGRHPENDRILFAAGHFRNGVLLAPATGEMVADMILGNPVKTEWIEAFKAERKEAVHR

SEQ ID NO: 5 - DNA, 1803 bp, Bacillus licheniformis
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccatgagaaagcgctatgatacgatcgtgatcggcggaggcatcatcgggacttccatcgcttatcatcttgcgaaagcggggaaaaaaacggccgtgtttgaaagcggggaagtcggcaaaaaagcgacgagcgcggctgcgggcatgctcggcgcccatgcggaatgcgataaacccggtacctttttcgaatttgccagagccagtcaaaaagcctataagcgcctgacgggggaattgaaagacataagcggcattgatatcagaaggcatgacggagggatattaaagctggcattttcagaaagcgaccgcgaacacctgatgcaaatgggtgcactggattctgtggaatggcttgaagctgatgaagtatataaattagaaccgaatgccggaaaaggaattctcggcgccaattttatcagggacgacgtccatgtcgaaccggctgcagtgtgcagggcatttgccaggggagcccggatgctcggtgcagatgtcttcgagtatacgcccgttttatcgatagaatccgaagcgggggccgtccgcgtcacgtccgcttccggcacagcggaggcggagcacgctgtcattgcaagcggcgtgtggagcggagctttgtttaaacaaatcggcttagacaaaaggttttaccccgtcaaaggggagtgcctgtccgtatggaatgacggcatctctttgacgagaacgctttaccatgatcattgctacatcgttccgcgccatagcggaaggcttgtcgtcggcgcgacgatgaagcccggggactggaatgagcagccggaacttggcggaatcgaggagttgatccgaaaagccaagtcgatgctccctgggattgaatccatgaaaatcgaccaatgctgggccgggttgcgtcccgaaacaggggatggcaatccgtatatcggaagacatcctgaaaatgaccggattttgtttgctgccggacatttcagaaacggcatcctcctggcacctgcaaccggtgagatgatggcagacatgatcctcggcaatccggtcaaaacagagtggatcgaggcgtttaaagcggagcgaaaggaggctgtgcacaggtga

SEQ ID NO: 6 - protein, 600 aa, Bacillus licheniformis
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSMRKRYDTIVIGGGIIGTSIAYHLAKAGKKTAVFESGEVGKKATSAAAGMLGAHAECDKPGTFFEFARASQKAYKRLTGELKDISGIDIRRHDGGILKLAFSESDREHLMQMGALDSVEWLEADEVYKLEPNAGKGILGANFIRDDVHVEPAAVCRAFARGARMLGADVFEYTPVLSIESEAGAVRVTSASGTAEAEHAVIASGVWSGALFKQIGLDKRFYPVKGECLSVWNDGISLTRTLYHDHCYIVPRHSGRLVVGATMKPGDWNEQPELGGIEELIRKAKSMLPGIESMKIDQCWAGLRPETGDGNPYIGRHPENDRILFAAGHFRNGILLAPATGEMMADMILGNPVKTEWIEAFKAERKEAVHR

SEQ ID NO: 7 - DNA, 1803 bp, Bacillus licheniformis
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccatgagaaagcgctatgatacgatcgtgatcggcggaggcatcatcgggacttccatcgcttatcatcttgcgaaagcggggaaaaaaacggccgtgtttgaaagcggggaagtcggcaaaaaagcgacgagcgcggctgcgggcatgctcagcgcccatcgggaatgcgataaacccggtacctttttcgaatttgccagagccagtcaaaaagcctataagcgcctgacgggggaattgagagacataagcggcattgatatcagaaggcatgacggagggatattaaagctggcattttcagaaagcgaccgcgaacacctgatgcaaatgggtgcactggattctgtggaatggcttgaagctgatgaagtatataaattagaaccgaatgccggaaaaggaattctcggcgccaattttatcagggacgacgtccatgtcgaaccggctgcagtgtgcagggcatttgccaggggagcccggatgctcggtgcagatgtcttcgagtatacgcccgttttatcgatagaatccgaagcgggggccgtccgcgtcacgtccgcttccggcacagcggaggcggagcacgctgtcattgcatgcggcgtgtggagcggagctttgtttaaacaaatcggcttagacaaaaggttttaccccgtcaaaggggagtgcctgtccgtatggaatgacggcatctctttgacgagaacgctttaccatgatcattgctacatcgttccgcgccatagcggaaggcttgtcgtcggcgcgacgatgaagcccggggactggaatgagcagccggaacttggcggaatcgaggagttgatccgaaaagccaagtcgatgctccctgggattgaatccatgaaaatcgaccaatgctgggccgggttgcgtcccgaaacaggggatggcaatccgtatatcggaagacatcctgaaaatgaccggattttgtttgctgccggacatttcagaaacggcgtcctcctggcacctgcaaccggtgagatggtggcagacatgatcctcggcaatccggtcaaaacagagtggatcgaggcgtttaaagcggagcgaaaggaggctgtgcacaggtga

SEQ ID NO: 8 - protein, 600 aa, Bacillus licheniformis
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSMRKRYDTIVIGGGIIGTSIAYHLAKAGKKTAVFESGEVGKKATSAAAGMLSAHRECDKPGTFFEFARASQKAYKRLTGELRDISGIDIRRHDGGILKLAFSESDREHLMQMGALDSVEWLEADEVYKLEPNAGKGILGANFIRDDVHVEPAAVCRAFARGARMLGADVFEYTPVLSIESEAGAVRVTSASGTAEAEHAVIACGVWSGALFKQIGLDKRFYPVKGECLSVWNDGISLTRTLYHDHCYIVPRHSGRLVVGATMKPGDWNEQPELGGIEELIRKAKSMLPGIESMKIDQCWAGLRPETGDGNPYIGRHPENDRILFAAGHFRNGVLLAPATGEMVADMILGNPVKTEWIEAFKAERKEAVHR

SEQ ID NO: 9 - DNA, 1416 bp, Bacillus licheniformis
atgggtcatcaccatcatcatcacgggtccctgcaggaggagaagcccaaggagggtgtgaagacagagaatgaccacatcaacctgaaggtggccgggcaggacggctccgtggtgcagttcaagatcaagaggcacacgccgctgagcaagctgatgaaggcctactgcgagaggcagggcttgtcaatgaggcagatcagattcaggttcgacgggcagccaatcaatgaaactgacactccagcacagctggagatggaggacgaggacaccatcgacgtgttccagcagcagacgggaggtatgagaaagcgctatgatacgatcgtgatcggcggaggcatcatcgggacttccatcgcttatcatcttgcgaaagcggggaaaaaaacggccgtgtttgaaagcggggaagtcggcaaaaaagcgacgagcgcggctgcgggcatgctcggcgcccatgcggaatgcgataaacccggtacctttttcgaatttgccagagccagtcaaaaagcctataagcgcctgacgggggaattgaaagacataagcggcattgatatcagaaggcatgacggagggatattaaagctggcattttcagaaagcgaccgcgaacacctgatgcaaatgggtgcactggattctgtggaatggcttgaagctgatgaagtatataaattagaaccgaatgccggaaaaggaattctcggcgccaattttatcagggacgacgtccatgtcgaaccggctgcagtgtgcagggcatttgccaggggagcccggatgctcggtgcagatgtcttcgagtatacgcccgttttatcgatagaatccgaagcgggggccgtccgcgtcacgtccgcttccggcacagcggaggcggagcacgctgtcattgcaagcggcgtgtggagcggagctttgtttaaacaaatcggcttagacaaaaggttttaccccgtcaaaggggagtgcctgtccgtatggaatgacggcatctctttgacgagaacgctttaccatgatcattgctacatcgttccgcgccatagcggaaggcttgtcgtcggcgcgacgatgaagcccggggactggaatgagcagccggaacttggcggaatcgaggagttgatccgaaaagccaagtcgatgctccctgggattgaatccatgaaaatcgaccaatgctgggccgggttgcgtcccgaaacaggggatggcaatccgtatatcggaagacatcctgaaaatgaccggattttgtttgctgccggacatttcagaaacggcatcctcctggcacctgcaaccggtgagatgatggcagacatgatcctcggcaatccggtcaaaacagagtggatcgaggcgtttaaagcggagcgaaaggaggctgtgcacaggtga

SEQ ID NO: 10 - protein, 471 aa, Bacillus licheniformis
MGHHHHHHGSLQEEKPKEGVKTENDHINLKVAGQDGSVVQFKIKRHTPLSKLMKAYCERQGLSMRQIRFRFDGQPINETDTPAQLEMEDEDTIDVFQQQTGGMRKRYDTIVIGGGIIGTSIAYHLAKAGKKTAVFESGEVGKKATSAAAGMLGAHAECDKPGTFFEFARASQKAYKRLTGELKDISGIDIRRHDGGILKLAFSESDREHLMQMGALDSVEWLEADEVYKLEPNAGKGILGANFIRDDVHVEPAAVCRAFARGARMLGADVFEYTPVLSIESEAGAVRVTSASGTAEAEHAVIASGVWSGALFKQIGLDKRFYPVKGECLSVWNDGISLTRTLYHDHCYIVPRHSGRLVVGATMKPGDWNEQPELGGIEELIRKAKSMLPGIESMKIDQCWAGLRPETGDGNPYIGRHPENDRILFAAGHFRNGILLAPATGEMMADMILGNPVKTEWIEAFKAERKEAVHR

SEQ ID NO: 11 - DNA, 1416 bp, Bacillus licheniformis
atgggtcatcaccatcatcatcacgggtccctgcaggaggagaagcccaaggagggtgtgaagacagagaatgaccacatcaacctgaaggtggccgggcaggacggctccgtggtgcagttcaagatcaagaggcacacgccgctgagcaagctgatgaaggcctactgcgagaggcagggcttgtcaatgaggcagatcagattcaggttcgacgggcagccaatcaatgaaactgacactccagcacagctggagatggaggacgaggacaccatcgacgtgttccagcagcagacgggaggtatgagaaagcgctatgatacgatcgtgatcggcggaggcatcatcgggacttccatcgcttatcatcttgcgaaagcggggaaaaaaacggccgtgtttgaaagcggggaagtcggcaaaaaagcgacgagcgcggctgcgggcatgctcagcgcccatcgggaatgcgataaacccggtacctttttcgaatttgccagagccagtcaaaaagcctataagcgcctgacgggggaattgagagacataagcggcattgatatcagaaggcatgacggagggatattaaagctggcattttcagaaagcgaccgcgaacacctgatgcaaatgggtgcactggattctgtggaatggcttgaagctgatgaagtatataaattagaaccgaatgccggaaaaggaattctcggcgccaattttatcagggacgacgtccatgtcgaaccggctgcagtgtgcagggcatttgccaggggagcccggatgctcggtgcagatgtcttcgagtatacgcccgttttatcgatagaatccgaagcgggggccgtccgcgtcacgtccgcttccggcacagcggaggcggagcacgctgtcattgcatgcggcgtgtggagcggagctttgtttaaacaaatcggcttagacaaaaggttttaccccgtcaaaggggagtgcctgtccgtatggaatgacggcatctctttgacgagaacgctttaccatgatcattgctacatcgttccgcgccatagcggaaggcttgtcgtcggcgcgacgatgaagcccggggactggaatgagcagccggaacttggcggaatcgaggagttgatccgaaaagccaagtcgatgctccctgggattgaatccatgaaaatcgaccaatgctgggccgggttgcgtcccgaaacaggggatggcaatccgtatatcggaagacatcctgaaaatgaccggattttgtttgctgccggacatttcagaaacggcgtcctcctggcacctgcaaccggtgagatggtggcagacatgatcctcggcaatccggtcaaaacagagtggatcgaggcgtttaaagcggagcgaaaggaggctgtgcacaggtga

SEQ ID NO: 12 - protein, 471 aa, Bacillus licheniformis
MGHHHHHHGSLQEEKPKEGVKTENDHINLKVAGQDGSVVQFKIKRHTPLSKLMKAYCERQGLSMRQIRFRFDGQPINETDTPAQLEMEDEDTIDVFQQQTGGMRKRYDTIVIGGGIIGTSIAYHLAKAGKKTAVFESGEVGKKATSAAAGMLSAHRECDKPGTFFEFARASQKAYKRLTGELRDISGIDIRRHDGGILKLAFSESDREHLMQMGALDSVEWLEADEVYKLEPNAGKGILGANFIRDDVHVEPAAVCRAFARGARMLGADVFEYTPVLSIESEAGAVRVTSASGTAEAEHAVIACGVWSGALFKQIGLDKRFYPVKGECLSVWNDGISLTRTLYHDHCYIVPRHSGRLVVGATMKPGDWNEQPELGGIEELIRKAKSMLPGIESMKIDQCWAGLRPETGDGNPYIGRHPENDRILFAAGHFRNGVLLAPATGEMVADMILGNPVKTEWIEAFKAERKEAVHR

(19) United States
(12) Patent Application Publication
(10) Pub. No.: US 2023/0242964 A1
(43) Pub. Date: Aug. 3, 2023

(54) BIODEGRADABLE BIOCHEMICAL SENSOR FOR DETERMINING THE PRESENCE AND/OR THE LEVEL OF PESTICIDES OR ENDOCRINE DISRUPTORS: METHOD AND COMPOSITION

(71) Applicants: SKILLCELL, BAlE MAHAULT (FR); CENTRE NATIONALE DE LA RECHERCHE SCIENTIFIQUE (CNRS), PARIS (FR)

(72) Inventors: Julien ESPEUT, SAINT MARTIN DE LONDRES (FR); Franck MOLINA, LES MATELLES (FR)

(21) Appl. No.: 17/415,070

(22) PCT Filed: Dec. 20, 2019
(86) PCT No.: PCT/EP2019/086838

§ 371 Date: Jun. 17, 2021

(30) Foreign Application Priority Data
Dec. 20, 2018 (EP) 18214973.2

(51) Int. Cl. C12Q 1/26; C12Q 1/48

(52) U.S. Cl. CPC C12Q 1/26; C12Q 1/48

(57) ABSTRACT

The present invention is directed to biodegradable biochemical sensor method to perform in a sample multiplex detection and/or quantification of pesticides and/or endocrine disruptors and to provide and logical integrated response to the user. This biochemical sensor is a vesicle encapsulating biochemical networks using enzymes capable of generating, inhibiting or activating specific measurable signal in presence of said target analytes. The biochemical network is able to provide an integrated logical final response to the user. The present invention also relates to a composition or kit comprising said biochemical sensor vesicle.

The present invention is directed to a method to detect and/or to quantify pesticides or endocrine disruptors in a sample using vesicle encapsulated biochemical reagents including 1 or more enzymes capable of generating or inhibiting specific measurable signal in presence of said target analyte. The present invention also relates to a composition or kit comprising said vesicle.

Pesticides are used to control and/or eliminate plant or animal pests and diseases. Pesticides can be classified as herbicides, insecticides, fungicides, or other types according to their purpose, and they involve different chemical compounds.

Pesticides can be classified by biological target, chemical structure, or safety profile. Because of the high toxicity of pesticides, environmental agencies have set maximum values for their contamination levels in drinking and surface water.

Depending on their aqueous solubility, pesticides either remain in the soil or enter surface waters and groundwater.

The conventional methods of pesticide residue analysis, especially for pesticide residues in vegetables and fruits, include spectrophotometry, nuclear magnetic resonance spectroscopy, thin layer chromatography, atomic absorption spectroscopy, gas chromatography, liquid chromatography, mass-spectrometry, fluorimetry and so on, among which gas chromatography and liquid chromatography coupled to mass-spectrometry are more commonly used due to advantages of favourable repeatability, sensitivity, and capability of determining pesticide type and concentration. Such methods have to be executed by following standard detection steps as well as by laboratory technicians equipped with the expertise conducting sample pre-treatment and performing analysis via instrumental operation. They offer powerful trace analysis with high reproducibility but these techniques involve extraction of large volumes of water, require extensive purification, and demand qualified personnel and expensive equipment.

In recent years, several methods for detecting enzyme inhibiting pesticides by means of biochemical reaction and electrochemistry technique have been developed, particularly using immobilized enzyme technology (see U.S. Pat. No. 6,406,876 (Gordon et al.; CN patent CN101082599 (Lin et al.)). Method for immobilizing enzyme on the electrode to determine pesticide concentration in aqueous solution via the degree of enzyme inhibition caused by pesticides has also been disclosed (see TW patent 1301541 (Wu et al.)). However, the methods of immobilizing enzyme are complicated and drawbacks of immobilized enzyme include high cost, complicated manufacturing process and stringent preservation conditions (see US patent US20150300976A1 (Wang et al.) for review).

Great progress has recently been made in applying nanomaterials to sensor and biosensor development. Owing to the properties afforded by the small size of nanomaterials their large surface-to-volume ratios; their physicochemical properties, composition, and shape; and their unusual target binding characteristics, these sensors can markedly improve the sensitivity and specificity of analyte detection. Said properties, together with the overall structural robustness of nanomaterials, make these materials highly amenable for use in various detection schemes based on diverse transduction modes (Nanomaterials for Sensing and Destroying Pesticides. Gemma Aragay et al., Chemical Reviews, 2012, 112, 5317-5338).

Patent document US Application US20150355154A1 (Tae Jung Park et al.) can be cited which discloses a sensor system capable of detecting organophosphorus pesticide residue by inducing the aggregation of gold nanoparticles.

Patent document CN102553497A can be also cited which discloses a preparation method of multifunctional compound-stamp nanospheres having both fluorescence and magnetism, and their application to the detection on pesticide residue by modification of fluorescence intensity of the multifunctional compound-stamp nanospheres before and after selective adsorption to pesticide molecules of a template.

Finally, the international patent application document WO 2017/178896A2 (Molina et al.) can be also cited which discloses biosynthetic devices for their use in disease diagnostic method, implementing encapsulated enzyme capable of reacting with the target compound which is desired to test in a sample.

Endocrine disruptors are also known to cause harmful effects to human through various exposure routes. These chemicals mainly appear to interfere with the endocrine or hormone systems. As importantly, numerous studies have demonstrated that the accumulation of endocrine disruptors can induce fatal disorders including obesity and cancer. (Yang O. et al., J Cancer Prev. 2015 March; 20(1): 12-24).

Endocrine disruptors can affect every level of the endocrine system. First, they can disrupt the action of enzymes involved in steroidogenesis. These enzymes can be inhibited, as can the enzymes involved in metabolism of oestrogens. For instance, some polychlorinated biphenyl (PCB) metabolites inhibit sulfotransferase, resulting in an increase of circulating estradiol (Kester M H et al., Endocrinology. 2000; 141:1897-1900). Other endocrine disruptors are known to promote adipogenesis. These include biphenyl A (BPA) organophosphate pesticides, monosodium glutamate, and polybrominated diphenyl ethers (PBDEs).

The present invention is to provide a method for detecting and/or quantifying the presence of a target analyte selected from the group of pesticides and endocrine disruptors residues, present in a sample, in a solution or at the surface of solid product, particularly present in environments or food, wherein said pesticide or endocrine disruptor target which is desired to be tested is known to be a substrate or an inhibitor of a specific enzyme activity.

In a preferred embodiment, the present invention relates to such a method for its use in the field of agronomic food, environment or health diagnosis, agronomic food and environment field being the more preferred.

For example, the glyphosate is an herbicide that inhibit the 5-enolpyruvylshikimate-3-phosphate (EPSP) synthase, a key enzyme in the shikimic acid pathway, which is involved in the synthesis of the aromatic amino acids. EPSP inhibition leads to depletion of the aromatic amino acids tryptophan, tyrosine, and phenylalanine that are needed for protein synthesis. Glyphosate resistant crops with an alternative EPSP enzyme have been developed that allow using glyphosate on these crops with no crop injury (http://herbicidesymptoms.ipm.ucanr.edu/MOA/EPSP synthase inhibitors/).

In a first aspect, the present invention is directed to a method to detect the presence, or to detect a relevant quantity, or the absence, and/or to quantify the amount of at least one target analyte in a sample, the method comprising the steps of: a) contacting the sample with a composition wherein:
said composition comprises biochemical elements forming a biochemical network encapsulated in one or in a set of micro- or nano-vesicles (named hereinafter vesicles) permeable or not to the target analyte, said biochemical network comprising as biochemical element at least one enzyme having as substrate or as inhibitor or as activator said target analyte which is desired to detect and/or to quantify, and wherein:
   i) the target analyte is selected from the group consisting of pesticides and/or endocrine disruptors,
   ii) the biochemical network is capable of:
generating at least one specific readable/measurable output signal only in presence, preferably given a chosen threshold, of the target analyte when said target analyte is a substrate of the enzyme of said biochemical network; or
inhibiting the specific readable/measurable output signal generated by said biochemical network only in presence of the target analyte when said target analyte is an inhibitor of the enzyme of said biochemical network, and,
   b) determining the rate and/or level of the specific readable/measurable output signal produced by the biochemical network, the rate and/or level obtained being correlated to the presence or the absence and/or the amount of the target analyte in the sample.

In the present description, the use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

Throughout the entire specification, including the claims, the word “comprise” and variations of the word, such as “comprising” and “comprises” as well as “have,” “having,” “includes,” and “including,” and variations thereof, means that the named steps, elements, or materials to which it refers are essential, but other steps, elements, or materials may be added and still form a construct within the scope of the claim or disclosure. When recited in describing the invention and in a claim, it means that the invention and what is claimed is considered to be what follows and potentially more. These terms, particularly when applied to claims, are inclusive or open-ended and do not exclude additional, unrecited elements or method steps.

By the term enzyme inhibitor, it is intended to designate a compound which reduces the rate of an enzyme catalysed reaction by interfering with the enzyme in some way. An enzyme inhibitor for example a molecule that binds to an enzyme and decreases its activity. The binding of an inhibitor can stop a substrate from entering the enzyme's active site and/or hinder the enzyme from catalyzing its reaction.

By the term enzyme activator, it is intended to designate a compound which increases the rate, activity or velocity of an enzyme. Generally, they are molecules that bind to the enzymes. Their actions are opposite to the effect of enzymes

By the term enzyme substrate, it is intended to designate a compound which reacts with an enzyme to generate a product. It is the material upon which an enzyme acts.

By biomolecular elements, it is intended to designate molecules that are present in living organisms, including large macromolecules such as proteins, carbohydrates, lipids, and nucleic acids, as well as small molecules such as primary metabolites, secondary metabolites, and natural products.

By vesicle, or micro- or nano-vesicle, it is intended to designate vesicle having a size (diameter) between 5 nm and 500 μm, preferably between 10 nm and 200 μm, more preferably between 25 nm and 50 μm. It is also intended to designate unilamellar or multilamellar vesicle having lipid membrane (liposome) or synthetic polymer or copolymer.

By biochemical element encapsulated, or internalized or included or contained in a vesicle, it is intended to designate biochemical element which can be encapsulated in the inner compartment of the vesicle but also encapsulated into the membrane (bi- or multi-lamellar membrane) or attached to the vesicle membrane (external or internal membrane).

In a preferred embodiment of the method of the present invention, said biomolecular elements are selected from synthetic, semi-synthetic biomolecular elements or isolated from naturally occurring biological systems.

In a more preferred embodiment said at least one biomolecular element(s) is selected from the group consisting of proteins, nucleic acids, preferably non-coding nucleic acids, and metabolites. Enzymes and metabolites are particularly more preferred biomolecular element(s).

These proteins, particularly the enzymes, when encapsulated in these particle systems exhibit a very good stability, and enhanced kinetics, even at room temperature and their activity can be then preserved during a long time at room temperature.

By the term target analyte, it is also intended to designate a class or a group of pesticides or endocrine disruptors which is desired to detect or to quantify in the sample, when all members of said class or group are acting like a substrate, like an inhibitor or like an activator of said enzyme which is encapsulated in the vesicle or set of vesicles.

The term “target analyte” generally refers herein to any molecule of pesticide or endocrine disruptor that is detectable with the method and the kit as described herein. Non-limiting examples of pesticide or endocrine disruptor targets that are detectable with the method and the kit as described herein include, but are not limited to, chemical or biochemical compounds.

By way of non-limiting example, pesticides are selected from the group consisting of insecticides, herbicides, fungicides. Preferred are pesticides which can act as substrate or as inhibitor of enzyme activity.

By way of non-limiting example, endocrin disruptors (EDs) are selected from the group consisting of:

### A) EDs Binding to Oestrogen Receptors, Agonist or Antagonist Effect

### i) Agonists (Estrogenic Effect)

Bisphenol-A; Phtalates

Polyphenols including isoflavones and genistein

Some UV-screens (benzophenone 2; cinnamate; camphor derivatives)

### ii) Antagonists (Antiandrogenic Effect)

Pesticides, fungicides, herbicides (linurone, procymidone, vinclocolin) dioxin

### B) EDs Having Effects on Enzymes

Fungicides (azoles): Synthesis inhibitors: Inhibition affected step of synthesis (sterol demethylase and chromatase)

Isoflavones: Inhibition of thyroid peroxidase

Polyphenols (isoflavones, genistein): Sulfatase increased decreased sulfo-transferase (from W. Wuttke et al., Hormones 2010, 9(1):9-15).

In a preferred embodiment, the present invention is directed to a method for detecting the presence or the absence, and/or to quantify the amount of at least one target analyte in a sample, the method comprising the steps of:

from a sample containing or susceptible to contain the target analyte;
a) contacting the sample with a composition comprising biochemical elements forming a biochemical network, said biochemical network comprising as biochemical element at least one enzyme having as substrate or as inhibitor or as activator said target analyte which is desired to be detected and/or quantified, and wherein:
at least one of said biochemical elements forming a biochemical network is encapsulated in one micro- or nano-vesicle (named vesicle) permeable or not to the target analyte; or
at least two of said biochemical elements forming a biochemical network are encapsulated in two distinct vesicles permeable or not to the target analyte, wherein:
   i) said at least one target analyte which is desired to be detected or quantified in the sample is selected from the group of pesticide or endocrine disruptor, more preferably selected from the group of pesticides. Preferred are pesticides which can act as substrate or as inhibitor of enzyme activity. The group consisting of insecticides, herbicides, fungicides being the most preferred.
   ii) the biochemical network is capable of:
generating at least one specific readable/measurable output signal only in presence of the target analyte when said target analyte is a substrate of the enzyme of said biochemical network; or
inhibiting the specific readable/measurable output signal generated by said biochemical network only in presence of the target analyte when said target analyte is an inhibitor of the enzyme of said biochemical network, and,
   b) determining the rate and/or level of the specific readable/measurable output signal produced by the biochemical network, the rate and/or level obtained being correlated to the presence or the absence and/or the amount of the target analyte in the sample.
   In a preferred embodiment, when it is desired to encapsulate enzyme in a vesicle or to facilitate the entrance of the enzyme in the vesicle, surfactant, hemolysins or porins can be used:
Surfactants can be used to facilitate the transfer of glyphosate through the vesicle membrane since the glyphosate does not pass the lipidic membranes easily. Polyoxyethylene amine like the POE hydrogenated tallow amide, POE (3) N-tallow trimethylene diamine, POE (15) tallow amine, POE (5) tallow amine, POE (2) tallow amine can be used.
Hemolysins or porins proteins can also be inserted in the membrane to facilitate the transfer of enzymes, substrates or molecule to detect (like glyphosate) through the vesicle membrane (Deshpande et al. 2015, NATURE COMMUNICATIONS|DOI: 10.1038/ncomms10447; Vamvakaki et al. 2007, Biosens Bioelectron. 2007 Jun. 15; 22(12):2848-53. Epub 2007 Jan. 16; Karamdad et al. 2015, Lab Chip, 2015, 15, 557).

In a preferred embodiment, the target analyte, pesticide and/or endocrine disruptor, is a substrate of at least one enzyme encapsulated in said vesicle or is a substrate of at least one enzyme which is contained in the composition but not encapsulated in said vesicle.

In an also preferred embodiment, the target analyte, pesticide and/or endocrine disruptor, is an inhibitor of the activity of at least one said enzyme encapsulated or not in said vesicle.

In a preferred embodiment, the sample susceptible to contain the target analyte is selected from the group consisting of fluid or solid material sample, preferably environmental material sample, vegetal material, water (like drinking water, beverage, waste water, river or sea water), food products, soil extracts, industrial material, food production, plant extract, physiologic fluid (urine, blood, sweat, vegetal sap, etc. . . . ) or tissue from living organism (mammal, plant, poultry, etc. . . . ).

Non-limiting examples of tissue of living organisms include soft tissue, hard tissue, skin, surface tissue, outer tissue, internal tissue, a membrane, foetal tissue and endothelial tissue.

When the sample is from a food source, non-limiting examples of food sources can be plant (preferably edible plant) grains/seeds, beverages, milk and dairy products, fish, shellfish, eggs, commercially prepared and/or perishable foods for animal or human consumption.

As mentioned above, the sample can be in an external environment, such a soil, water ways, sludge, commercial effluent, and the like.

By sample, it is intended to particularly designate a sample of a material suspected of containing the analyte(s) of interest, which material can be a fluid or having sufficient fluidity to flow through or to be in contact with the vesicle of the composition implemented in the method of the present invention. The fluid sample can be used as obtained directly from the source or following a pre-treatment so as to modify its character. Such samples can include human, animal, vegetal or man-made samples as listed above but non-limited to. The sample can be prepared in any convenient medium which does not interfere with the assay. Typically, the sample is an aqueous solution or biological fluid, or the surface of a solid material.

Thus, said sample, can also designate the surface of a solid material suspected of containing the analyte(s) of interest, which solid material can be porous or non-porous and can be selected from made-man material, food products, plants, seeds, fruits and the like. In this case, the composition implemented in the method of the present invention and comprising a vesicle can be directly applied on the surface of this solid material, for example with composition of the present invention in the form of porous gel, like porous polymeric beads (for example agarose, alginate, polyvinyl-alcohol, dextran, acrylamide polymer derivatives beads) wherein the vesicles of the composition are retained.

In a preferred embodiment, the method of the present invention is characterized in that the presence or relevant quantity and/or amount of the target analyte is detected by a signal associated to an agent selected from the group consisting of a colorimetric agent, an electron transfer agent, an enzyme, a fluorescent agent, agent which provide said detectable or quantifiable signal correlated to the presence and/or the amount of the target analyte.

The present invention is also directed to a method to detect the presence or the absence, and/or to quantify the amount of at least two different target analytes in a sample wherein said different analytes are either substrates or inhibitors or activators of the same at least one biochemical network enzyme encapsulated or not encapsulated in the vesicle.

Indeed, the presence of two distinct analytes acting on the same vesicle encapsulated enzyme or not encapsulated enzyme or on the same biochemical network enzyme (as substrate or as inhibitor), can amplify the emitted signal.

For Example, (see Example 6, FIGS. 6, 7 and 12), the detection of a first target analyte (i.e. glyphosate) and a second target (i.e. glycine) can be detected or quantified separatively by the same biochemical network used in the method of the present invention. Moreover, using the same biochemical network according to the present invention, the two target analytes can be detected and/or quantified in the same time (see FIG. 12).

One of the advantages of the method of the present invention is to reduce or to remove the background noise usually present and which cause difficulties when different biochemical elements or biochemical network are using in a method of detection or quantification of a compound.

Using in the same device or composition, biochemical element which are in solution, encapsulated in vesicle or trapped in a gel matrix or in a solid surface allow important reduction of these background noises.

When it is necessary, for example when the specific signal cannot be directly readable by visual reading, the signal can be detected or quantified by colorimetric measurement, fluorescence, spectroscopy (i.e. infra-red, Raman), chemical compound or particle (electron) production.

In a preferred embodiment of the method of the present invention, said output signal which is capable of generating by said biochemical network is selected from a biological, chemical, electronic or photonic signal, preferably a readable and, optionally, measurable physicochemical output signal.

Among the signal which can be used as output signal, we can cite particularly and for example colorimetric, fluorescent, luminescent or electrochemical signal. These examples are not intended to limit the output signal which can be used in the present invention. Their choice mostly depends on assay specifications, in terms of sensitivities or technical resources. Importantly, colorimetric outputs are human readable, a property of interest for integration into low-cost, easy-to-use point of care devices, while for example luminescent signals offer ultrahigh sensitivities and wide dynamic range of detections. However, instead of measuring traditional end point signals, other biosensing frameworks exist, and can be achieved thanks to properties inherent to biological systems. It is thus possible to define different modes of readout, such as linear, frequency, or threshold, or multivalued modes of detection.

In another aspect, the method of the present invention can be used for detecting and/or to quantify the presence of two different target analytes in a same sample and wherein the composition implemented in the method comprises two different sets of biochemical elements forming two distinct biochemical networks encapsulated in the same or in at least two distinct vesicles or set of vesicles permeable or not to both of the target analytes, each of said biochemical elements comprising at least a different enzyme having as substrate or as inhibitor or as activator only one of the target analytes which are desired to detect and/or to quantify. In this case, the composition implemented in the method of the present invention contains at least two different sets of biochemical elements, each of them forming a different biochemical network generating a different readable/measurable output signal, said two different sets of biochemical elements being encapsulated in the same vesicle or in a different set of vesicles, or at least one of the biochemical elements forming the biochemical network and for each of the two distinct biochemical network are encapsulated in the same vesicle or in a different set of vesicles.

So, the present invention is also directed to a method to detect the presence or the absence, and/or to quantify the amount of at least two different target analytes in a sample, wherein:
said different analytes are substrates or inhibitors of two distinct biochemical network enzymes and wherein:
at least and for each of said two distinct biochemical networks, one of the biochemical elements forming said biochemical network is encapsulated in a vesicle permeable or not to the target analyte; or
at least two of said biochemical elements forming a biochemical network are encapsulated in two distinct vesicles permeable or not to the target analyte; and
said different biochemical networks (interconnected or not) generating a different readable/measurable output signal.

By “interconnected biochemical networks” it is intended to designate here that the two different biochemical networks could have common biochemical elements or a same common step or part of the network.

In a preferred embodiment, the method of the present invention is characterized in that the pesticide or endocrine disruptor which is desired to detect and/or to quantify is a biochemical element of the biochemical network which can produce in one step, or more, a specific readable/measurable output signal, said biochemical element being preferably selected from the group consisting of macromolecule, peptide, protein, metabolite, enzyme, nucleic acid, metal ion.

More preferably, the pesticide or endocrine disruptor which is desired to detect and/or to quantify are selected from the group consisting of:

a) pesticide or an endocrine disruptor molecule which is a specific substrate of an enzyme activity, activity which can produce in one step, or more, a specific readable/measurable output signal; or

b) pesticide or an endocrine disruptor molecule which is a specific inhibitor of an enzyme activity, activity which can produce in one step, or more, a specific readable/measurable output signal; or

c) pesticide or an endocrine disruptor molecule which is a specific activator of an enzyme activity, activity which can produce in one step, or more, a specific readable/measurable output signal.

In a more preferred embodiment, the pesticide or endocrine disruptor molecule which is a specific substrate of an enzyme activity, activity which can produce in one step, or more, a specific readable/measurable output signal associated with the present and/or amount of this target analyte in the sample is selected from the group consisting of the glyphosate (which is a substrate for the glycine/glyphosate oxidase enzyme), and chlordecone (chlordecone reductase substrate).

In an also more preferred embodiment, the pesticide or an endocrine disruptor molecule which is a specific inhibitor of an enzyme activity, activity which can produce in one step, or more, a specific readable/measurable output signal associated with the present and/or amount of this target analyte in the sample is selected from the group consisting of: Glyphosate (EPSPS inhibitor), chlordecone (Oestrogen receptors interfering agent), carbamates (Acetyl Cholinesterase inhibitor), succinate dehydrogenase inhibitors fungicides (SDHI fungicides). Are preferred the SDHI fungicides selected from the group of Oxathiin-carboxamide, Phenyl-Benzamides, Thiazole-carboxyamaide, Furan-carboxamide, Pyridine-carboxamide, Pyrazole-carboxamide and Pyridinyl-ethyl-benzamide. Neonicotinoids (inhibitors of acetylcholine receptors activity) preferably selected from the group of chloropyridinyl, trifluoropyridinyl, chlorothiazolyl, tetrahydrofuranyl, phenylpyrazole.

Others fungicides such as AnilinoPyrimidines (AP) Fungicides, Carboxylic Acid Amides (CAA) Fungicides and Sterol Biosynthesis Inhibitor's (SBI) can be cited which are also specific inhibitors of an enzyme activity (see the web site http://www.frac.info/working-group/ for complete information about these compounds).

The pesticide or an endocrine disruptor molecule selected from the group consisting of Neonicotinoid, Organochlorides, dioxine (PCDD), polychlorobiphenyl (PCB), 17-beta oestradiol, 17-alpha ethylene oestradiol, bisphenol (PBDE), phthalates, heavy metal (Cr, Mn, Pb, Li, Hg, etc.) are also preferred.

In an also more preferred embodiment, the elements forming the biochemical network comprises at least encapsulated in a vesicle at least one enzyme selected from the group consisting of Glycine/Glyphosate oxidase (EC 1.4.3.19), acetylcholinesterase (EC3.1.1.7), 5-enolpyruvylshikimate-3-phosphate (EPSP) synthase (EC 2.5.1.19) and succinate dehydrogenase (EC 1.3.5.1).

In another more preferred embodiment, the at least pesticide or an endocrine disruptor target analyte which is desired to detect and/or to quantify in the sample is the glyphosate.

In a more preferred embodiment, the present invention is directed to a method according to the present invention, for the detection of the presence or the absence, and/or to quantify the amount of at least the glyphosate as target analyte in a sample, said method comprising the steps of:

from a sample containing or susceptible to contain glyphosate;

a) contacting the sample with a composition comprising biochemical elements forming a biochemical network, said biochemical network comprising as biochemical element at least one enzyme having as substrate or as inhibitor or as activator said target analyte which is desired to be detected and/or quantified, and wherein:
at least one of said biochemical elements forming a biochemical network is encapsulated in one vesicle permeable or not to the target analyte; or
at least two of said biochemical elements forming a biochemical network are encapsulated in two distinct vesicles permeable or not to the target analyte, wherein:
   i) the biochemical network is capable of:
generating at least one specific readable/measurable output signal only in presence of the target analyte when said target analyte is a substrate of the enzyme of said biochemical network; or
inhibiting the specific readable/measurable output signal generated by said biochemical network only in presence of the target analyte when said target analyte is an inhibitor of the enzyme of said biochemical network, and,
   b) determining the rate and/or level of the specific readable/measurable output signal produced by the biochemical network, the rate and/or level obtained being correlated to the presence or the absence and/or the amount of glyphosate in the sample.

In a preferred embodiment, the biochemical elements forming the biochemical network comprises at least encapsulated or not in a vesicle the glycine/glyphosate oxidase enzyme (EC 1.4.3.19).

In a preferred embodiment said glycine/glyphosate oxidase enzyme is the native (or wild type/WT) glycine/glyphosate oxidase which can be obtained as recombinant protein.

In a more preferred embodiment said glycine/glyphosate oxidase enzyme comprises a tag which is fused to the glycine/glyphosate oxidase enzyme, particularly in order to enhance the recombinant expression and its solubility compared with native sequences (Jeffrey G. Marblestone et al. (Protein Sci. 2006 January; 15(1): 182-189)).

Among the tag which can be used but non-limiting to, maltose-binding protein (MBP), Chitin Binding Protein (CBP), glutathione S-transferase (GST), thioredoxin (TRX), NUS A, ubiquitin (Ub), and SUMO (small ubiquitin-related modifier) tags can be cited.

Tags comprising SUMO and GST are tags which are particularly preferred.

In an also more preferred embodiment said glycine/glyphosate oxidase enzyme is the glycine oxidase (GO) from the marine bacteria Bacillus licheniformis ((BliGO) which has been cloned and which shows 62% similarity to the standard GO from Bacillus subtilis (see Characterization and directed evolution of BliGO, a novel glycine oxidase from Bacillus licheniformis. Zhang K et al. (Enzyme Microb Technol. 2016 April; 85: 12-8.). Homolog sequence having at least 60%, 70%, preferably 75%, 80%, 85%, 90% or 95% identity (using for example the standard BLAST-P or BLAST-N software for aligment) with the BliGO WT protein sequence and preferably exhibiting at least GO activity, preferably at least 50% of BliGO WT GO activity in the same conditions of activity test, are also preferred.

Are preferred the GST-BliGO (native/WT) having the DNA sequence SEQ ID NO:5 or the amino acids sequence SEQ ID NO:6 (see FIG. 16) and the SUMO-BliGO (native/WT) having the DNA sequence SEQ ID NO:9 or the amino acids sequence SEQ ID NO:10 (see FIG. 18), or homolog tagged BliGO sequences thereof as defined above wherein the BliGO sequence exhibits at least 70%, preferably 75%, 80%, 85%, 90% or 95% identity with the WT BliGO.

In a more preferred embodiment said glycine/glyphosate oxidase enzyme is the mutated glycine oxidase (GO) from the marine bacteria Bacillus licheniformis ((BliGO)—SCF4 genetically modified and containing 6 single amino-acids mutation compared to the wild type version BliGO-WT has been cloned and which shows 62% similarity to the standard GO from Bacillus subtilis (see Characterization and directed evolution of BliGO, a novel glycine oxidase from Bacillus licheniformis. Zhang K et al. (Enzyme Microb Technol. 2016 April; 85:12-8.).

Are also preferred the GST-BliGO_Mut having the DNA sequence SEQ ID NO:7 or the amino acids sequence SEQ ID NO:8 (see FIG. 17) and the SUMO-BliGO_Mut having the DNA sequence SEQ ID NO:11 or the amino acids sequence SEQ ID NO:12 (see FIG. 19).

In a preferred embodiment, the biochemical elements forming the biochemical network further comprises in addition to glycine/glyphosate oxidase (EC 1.4.3.19), at least encapsulated in the same particle or in another vesicle a peroxidase, preferably the horseradish-peroxidase (HRP) enzyme (EC.1.11.17), and a substrate of a peroxidase which can be oxidized, preferably 0-dianisidine, pyrogallol, or amplex red, 2,2′-azino-bis(3-ethylbenzothiazoline-6-sulphonic) acid (ABTS), o-Phenylenediamine (OPD), 3,3′-Diaminobenzidine (DAB), 3-Amino-9-ethylcarbazole (AEC), 3,3′,5,5′-Tetramethylbenzidin (TMB), homovanillic acid, Tyramin or Luminol.

In another more preferred embodiment, the pesticide or an endocrine disruptor target analyte which is desired to detect and/or to quantify in the sample is the glyphosate, and wherein the biochemical elements forming the biochemical network comprises at least encapsulated in a vesicle the 5-enolpyruvylshikimate-3-phosphate (EPSP) synthase (EPSPS) enzyme (EC 2.5.1.19), 3-phospho-shikimate and phosphoenolpyruvate (PEP).

In a preferred embodiment, the biochemical elements forming the biochemical network further comprises in addition to the 5-enolpyruvylshikimate-3-phosphate (EPSP) synthase (EPSPS) enzyme (EC 2.5.1.19), 3-phospho-shikimate and phosphoenolpyruvate (PEP), at least encapsulated in the same particle or in another one or more particles the chorismate synthase enzyme (EC.4.2.3.5), the chorismate lyase enzyme (EC.4.1.3.40), lactate dehydrogenase enzyme (EC 1.1.1.27) and its NADH substrate.

In another more preferred embodiment, the pesticide or an endocrine disruptor target analyte which is desired to detect and/or to quantify in the sample is the glyphosate, and wherein the biochemical elements forming the biochemical network comprises at least encapsulated in a vesicle the 5-enolpyruvylshikimate-3-phosphate (EPSP) synthase (EPSPS) enzyme (EC 2.5.1.19), 3-phospho-shikimate and phosphoenolpyruvate (PEP), and in the same particle or in another one or more particles, the purine-nucleoside phosphorylase enzyme (EC.2.4.2.1.) and its inosine substrate, the xanthine oxidase enzyme (EC. 1.17.3.2), a peroxidase, preferably the horse radish-peroxidase (HRP) enzyme (EC.1.11.17), and a substrate of a peroxidase which can be oxydized, preferably 0-dianisidine, pyrogallol, or amplex red, 2,2′-azino-bis(3-ethylbenzothiazoline-6-sulphonic) acid (ABTS), o-Phénylènediamine (OPD), 3,3′-Diaminobenzidine (DAB), 3-Amino-9-éthylcarbazole (AEC), 3,3′,5,5′-Tétraméthylbenzidin (TMB), homovanillic acid, Tyramin or Luminol.

In a preferred embodiment the vesicles are selected from the group consisting of unilamellar or multilamellar vesicles, preferred are lipid vesicles, liposomes or self-assembled phospholipids, or vesicles formed from synthetic polymers or copolymers, said vesicles having preferably an average diameter between 0.01 μm to 500 μm, preferably between 0.01 μm to 100 μm, more preferably between 0.05 μm to 50 μm or between 0.05 μm to 10 μm.

For example, but non-limited to, the biochemical elements of the composition implemented in the method of the present invention can be compartmentalized/confined or encapsulated in a compartment, for example in a vesicular system or in any other kind of compartment, having a vesicular nature or not such but not limited to a porous gel, a porous polymeric bead, assembled phospholipids such as liposome, synthetic copolymers.

In the present description, is also used the wording “confined” or “compartmentalized” for “encapsulated”, which have the same meaning.

In a preferred embodiment the vesicles of the composition implemented in the method of the present invention are trapped in a porous polymeric gel, preferably selected from the group of porous polymeric gel consisting of alginate, chitosan, PVP (polyvinylpyrrolidone), PVA (polyvinyl-alcohol), agarose, sephadex, sepharose, sephacryl and mixture thereof.

For example, the method to detect the presence or the absence, and/or to quantify the amount of at least one target analyte in a sample, according to the present invention, comprises the steps of:

a) bringing into contact the composition implemented in the method of the present invention with a sample susceptible to contain said target analyte compound, to generate a mixture,

b) incubating said mixture in conditions adapted for the performance of at least one biochemical reaction, to generate at least said output signal, preferably readable/measurable physicochemical output signal, wherein said output signal being indicative of the presence, or a relevant quantity of, and/or the level of the compound which is desired to analyze in said sample.

c) detecting or measuring the output signal generated at step b), and

d) determining, form the signal generated/measured in step c), the presence and/or the level of said compound.

It is also to be understood that, in certain embodiments of the method of the present invention, the method can detect target analyte(s) over desired time duration. The duration can be a first pre-determined time interval and a least a second pre-determined time interval that are calculated. In certain embodiments, an analyte correlation value is calculated during the test time interval.

In a second aspect, the present invention is directed to a composition to detect the presence or the absence, and/or to quantify the amount of at least one target analyte in a sample said composition comprising biochemical elements forming a biochemical network encapsulated in one or in a set of vesicles permeable or not permeable to the target analyte, said biochemical network comprising as biochemical element at least one enzyme having as substrate or as inhibitor said target analyte which is desired to detect and/or to quantify, and wherein:

i) the target analyte is selected from the group consisting of pesticides and/or endocrine disruptors,
ii) the biochemical network is capable of either:

a) generating at least one specific readable/measurable output signal only in presence of the target analyte when said target analyte is a substrate of the enzyme of said biochemical network; or

b) inhibiting the specific readable/measurable output signal generated by said biochemical network only in presence of the target analyte when said target analyte is an inhibitor of the enzyme of said biochemical network.

In a preferred embodiment the vesicle is permeable to the target analyte.

In a preferred embodiment the composition according to the present invention comprises a vesicle or a set of vesicles having the characteristics as defined above in the composition implemented for the method of the present invention.

In a more preferred embodiment, said composition of the present invention comprises biochemical elements forming a biochemical network encapsulated in one or in a set of vesicles permeable or not permeable to the target analyte, said biochemical network comprising:

A) as biochemical elements, one of the biochemical elements selected from the group of:
glycine/glyphosate oxidase (EC 1.4.3.19), acetylcholinesterase (EC3.1.1.7), 5-enolpyruvylshikimate-3-phosphate (EPSP) synthase (EC 2.5.1.19) and succinate dehydrogenase (EC 1.3.5.1), and, optionally,
i) in addition to glycine/glyphosate oxidase (EC 1.4.3.19), at least a peroxidase, preferably the horseradish-peroxidase (HRP) enzyme (EC.1.11.17), and, optionally, a substrate of a peroxidase which can be oxidized, preferably 0-dianisidine, pyrogallol, or amplex red, 2,2′-azino-bis(3-éthylbenzothiazoline-6-sulphonic) acid (ABTS), o-Phenylenediamine (OPD), 3,3′-Diaminobenzidine (DAB), 3-Amino-9-éthylcarbazole (AEC), 3,3′,5,5′-Tétraméthylbenzidin (TMB), homovanillic acid, Tyramin or Luminol, and, optionally,
ii) in addition to the 5-enolpyruvylshikimate-3-phosphate (EPSP) synthase (EPSPS) enzyme (EC 2.5.1.19), at least 3-phospho-shikimate and phosphoenolpyruvate (PEP), and, optionally in addition:
   ii)a) chorismate synthase enzyme (EC.4.2.3.5), chorismate lyase enzyme (EC.4.1.3.40), lactate dehydrogenase enzyme (EC 1.1.1.27) and its NADH substrate, or
   ii)b) the purine-nucleoside phosphorylase enzyme (EC.2.4.2.1.) and its inosine substrate, the xanthine oxidase enzyme (EC. 1.17.3.2), a peroxidase, preferably the horse radish-peroxidase (HRP) enzyme (EC.1.11.17), and a substrate of a peroxidase which can be oxydized, preferably 0-dianisidine, pyrogallol, or amplex red, 2,2′-azino-bis(3-ethylbenzothiazoline-6-sulphonic) acid (ABTS), o-Phénylènediamine (OPD), 3,3′-Diaminobenzidine (DAB), 3-Amino-9-éthylcarbazole (AEC), 3,3′,5,5′-Tétraméthylbenzidin (TMB), homovanillic acid, Tyramin or Luminol;
      and
      B) as vesicles, the vesicles selected from the group consisting of:
unilamellar or multilamellar vesicles, preferred are lipid vesicles, liposomes or self-assembled phospholipids, or vesicles formed from synthetic polymers or copolymers, said vesicles having preferably an average diameter between 0.01 μm to 500 μm, preferably between 0.01 μm to 100 μm, more preferably between 0.05 μm to 50 μm or between 0.05 μm to 10 μm; and/or
vesicle having a vesicular nature or not such as, but not limited to, a porous gel, a porous polymeric bead, assembled phospholipids such as liposome, synthetic copolymers.

In a more preferred embodiment the vesicles of the composition of the present invention are trapped in a porous polymeric gel, preferably selected from the group of porous polymeric gel consisting of alginate, chitosan, PVP (polyvinylpyrrolidone), PVA (polyvinyl-alcohol), agarose, sephadex, sepharose, sephacryl and mixture thereof.

In a more preferred embodiment the composition of the present invention is directed to a composition comprising biochemical elements forming a biochemical network encapsulated or not in one or in a set of vesicles permeable or not to the target analyte, said biochemical network comprising as biochemical element at least one enzyme selected from the group of:
glycine/glyphosate oxidase (EC 1.4.3.19), preferably the native (wild type/WT) glycine/glyphosate oxidase which can be obtained as recombinant protein, or homolog sequence thereof having at least 70% identity with the WT protein sequence and exhibiting glycine/glyphosate oxidase activity;
5-enolpyruvylshikimate-3-phosphate (EPSP) synthase (EC 2.5.1.19);
the glycine oxidase (GO) from the marine bacteria Bacillus licheniformis ((BliGO) which has been cloned and which shows at least 62% similarity to the standard GO from Bacillus subtilis, or homolog BliGO sequence thereof having at least 70% identity with the BliGO WT protein sequence and exhibiting GO activity;
the mutated glycine oxidase (GO) from the marine bacteria Bacillus licheniformis ((BliGO)_SCF4 (also named BliGO-Mut) genetically modified and containing 6 single amino-acids mutation compared to the wild type version BliGO-WT;
a tagged glyphosate oxidase enzyme, preferably with a tag selected from the group consisting of maltose-binding protein (MBP), Chitin Binding Protein (CBP), glutathione S-transferase (GST), thioredoxin (TRX), NUS A, ubiquitin (Ub), and SUMO (small ubiquitin-related modifier) tags, preferably SUMO and GST tags; and
the GST-BliGO (native/WT) having the DNA sequence SEQ ID NO:5 or the amino acids sequence SEQ ID NO:6;
the SUMO-BliGO (native/WT) having the DNA sequence SEQ ID NO:9 or the amino acids sequence SEQ ID NO:10;
the GST-BliGO-Mut having the DNA sequence SEQ ID NO:7 or the amino acids sequence SEQ ID NO:8 and the SUMO-BliGO-Mut having the DNA sequence SEQ ID NO:11 or the amino acids sequence SEQ ID NO:12 and
   homolog tagged BliGO sequences thereof as defined above wherein the BliGO sequence exhibits at least 70%, and optionally
      one vesicle as defined above, and wherein at least one biochemical elements forming a biochemical network is encapsulated in a vesicle and or trapped in a gel matrix.

In a third aspect, the present invention is directed to a kit or a device to detect the presence, or the presence of a relevant quantity, or the absence, and/or to quantify the amount of at least one target analyte in a sample said kit comprising a container containing the composition according to the present invention or as defined as defined above in the composition implemented for the method of the present invention, wherein the vesicles of said composition are trapped in a porous polymeric gel, preferably selected from the group consisting of porous polymeric gel, preferably selected from the group consisting of alginate, chitosan, PVP (polyvinylpyrrolidone), PVA (polyvinyl-alcohol), agarose, sephadex, sepharose, sephacryl, and mixture thereof.

It is to be understood that both the foregoing general description and the following detailed description are exemplary and explanatory only and are not intended to limit the scope of the current teachings. In this application, the use of the singular includes the plural unless specifically stated otherwise.

Those of ordinary skill in the art will realize that the following detailed description of the embodiments is illustrative only and not intended to be in any way limiting. Other embodiments will readily suggest themselves to such skilled persons having the benefit of this disclosure. Reference to an “embodiment,” “aspect,” or “example” herein indicate that the embodiments of the invention so described may include a particular feature, structure, or characteristic, but not every embodiment necessarily includes the particular feature, structure, or characteristic. Further, repeated use of the phrase “in one embodiment” does not necessarily refer to the same embodiment, although it may.

The following examples, the figures and the legends hereinafter have been chosen to provide those skilled in the art with a complete description in order to be able to implement and use the present invention These examples are not intended to limit the scope of what the inventor considers to be its invention, nor are they intended to show that only the experiments hereinafter were carried out.

Other characteristics and advantages of the invention will emerge in the remainder of the description with the Examples and Figures, for which the legends are given hereinbelow.

### FIGURE LEGENDS

FIG. 1: Schematic representation of the glycine/glyphosate biochemical network #1. The network comprises the GST-BliGO Mut #1 enzyme, the HRP enzyme and the Amplex Red or dianisidine for the colorimetric or fluorescent readout.

FIGS. 2A-2B: Schematic representation of the EPSP synthase biochemical network #2A and #2B for glyphosate detection.

The network #2A (FIG. 2A) comprises the EPSP synthase enzyme, the Chorismate synthase enzyme, the Chorimsate lyase enzyme, the lactate dehydrogenase enzyme and the NADH for the absorbance or fluorescence detection.

The network #2B comprises the EPSP synthase enzyme, the Purine-nucleoside phosphorylase enzyme, the Xanthine oxidase enzyme, the HRP enzyme and the Amplex Red or O-dianisidine for the colorimetric or fluorescent readout.

FIG. 3: Microfluidic process for vesicle formation (From Courbet et al. Mol. Sys. Biol. 2018, 14(4):e7845. FIG. 4A))

FIGS. 4A-4B:

FIG. 4A: Absorbance glyphosate detection by the Glycine/Glyphosate oxidase network #1. Detection of Glyphosate ranging from 0 to 10 mM.

FIG. 4B: Glycine/Glyphosate oxidase enzyme catalytic activity analysis.

FIGS. 5A-5B:

FIG. 5A: Fluorescence Phosphoenol pyruvate (PEP) detection by the Glycine/Glyphosate oxidase network #2B. Detection of PEP ranging from 0 to 100 μM. FIG. 5B: EPSP synthase enzyme catalytic activity analysis.

FIG. 6: Schematic representation of the Glycine biochemical network #3 for glycine detection. The network comprises the Bacillus Subtilis Glycine oxidase H244K enzyme, the HRP enzyme and the Amplex Red or O-dianisidine for the colorimetric or fluorescent readout.

FIG. 7: Schematic representation of the Glyphosate OR Glycine biochemical network #4 for glyphosate and glycine detection. The network comprises the GST-BliGO Mut #1 enzyme, the Bacillus Subtilis Glycine oxidase H244K enzyme, the HRP enzyme and the Amplex Red or O-dianisidine for the colorimetric or fluorescent readout.

FIG. 8A-8B: Fluorescence (upper part) and colorimetric (lower part) glyphosate detection by the Glycine/Glyphosate oxidase network #1. (A-Left) Detection of Glyphosate ranging from 0 to 2 mM in Tris buffer 50 mM pH 7.5. (B-Right) Detection of Glyphosate ranging from 0 to 2 mM in barley seeds extracted in Tris buffer 50 mM pH 7,5.

FIG. 9A-9B: Fluorescence (upper part) and colorimetric (lower part) glyphosate detection by the Glycine/Glyphosate oxidase network #1 integrated in vesicles. (A-Left) Detection of Glyphosate ranging from 0 to 2 mM in Tris buffer 50 mM pH 7.5. (B-Right) Detection of Glyphosate ranging from 0 to 2 mM in barley seeds extracted in Tris buffer 50 mM pH 7,5.

FIG. 10: Colorimetric glyphosate detection by the Glycine/Glyphosate oxidase network #1 integrated in alginate beads. (Upper part) Detection of Glyphosate ranging from 0 to 4 mM in Tris buffer 50 mM pH 7,5. (Lower part) Detection of Glyphosate ranging from 0 to 4 mM in barley seeds extracted in Tris buffer 50 mM pH 7,5.

FIG. 11: Fluorescence (upper part) and colorimetric (lower part) glycine detection by the Glycine/Glyphosate oxidase network #3. (Left) Detection of glycine ranging from 0 to 1 mM in Tris buffer 50 mM pH 7,5. Note the absence of detection of the glyphosate at 100 μM.

FIG. 12: Fluorescence glyphosate and glycine detection by the Glycine/Glyphosate oxidase network #4. (Upper part) Kinetic of glyphosate AND/OR glycine degradation by the network. Glyphosate and glycine were present at 1 mM concentration. (Lower part) Glycine/Glyphosate oxidase network #4 logic-gate (OR) response to glycine AND/OR glyphosate presence.

FIG. 13: Fluorescence glyphosate detection by the EPSP synthase network #2B. Kinetic of glyphosate degradation by the network. Detection of glyphosate ranging from 0 to 1 mM.

FIG. 14: BliGO_WT (native) Protein: DNA (SEQ ID NO:1) and amino acids (SEQ ID NO:2) sequence

FIG. 15: BliGO_Mut Protein: DNA (SEQ ID NO:3) and amino acids (SEQ ID NO:4) sequence

FIG. 16: GST-BliGO_WT (native) Protein: DNA (SEQ ID NO:5) and amino acids (SEQ ID NO:6) sequence

FIG. 17: GST-BliGO_Mut Protein: DNA (SEQ ID NO:7) and amino acids (SEQ ID NO:8) sequence

FIG. 18: SUMO-BliGO_WT Protein: DNA (SEQ ID NO:9) and amino acids (SEQ ID NO:10) sequence

FIG. 19: SUMO-BliGO_Mut Protein: DNA (SEQ ID NO:11) and amino acids (SEQ ID NO:12) sequence.

### Example 1: Study Design—Setup of the Biochemical Networks

Different Biochemical Networks have been designed to detect the presence of different pesticides and/or endocrine disruptors. One originality of our invention resides in the fact that different biochemical networks can be plugged together to allow the detection of different analytes and lead to the delivery of a single output signal if necessary.

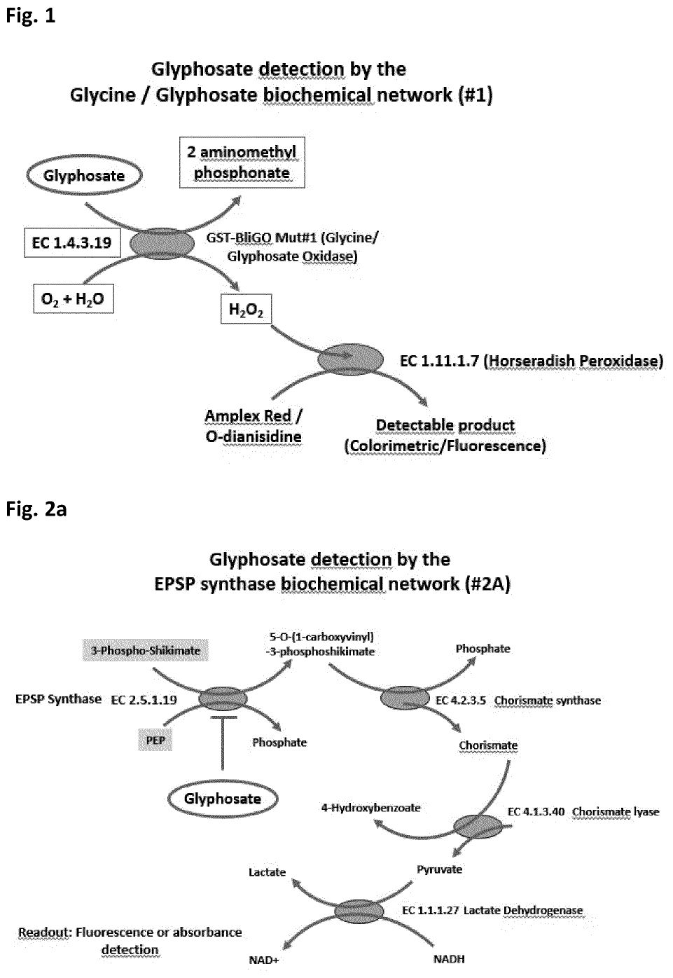

For the specific detection of the glyphosate pesticide we designed two detection systems that can be combined together to improve the specificity of the output signal:
1. The first network uses the ability of the enzyme Glycine/Glyphosate oxidase to metabolize the Glyphosate (FIG. 1).

In a first example, this first network comprises:

a) the enzyme Glycine/Glyphosate oxidase, the enzyme Horseradish Peroxidase and the O-Dianisidine dihydrochloride. In the presence of Glyphosate, 2-amino phosphonate and H₂O₂ are produced by the Glycine/Glyphosate oxidase. Afterward, the H₂O₂ is co-processed with the O-dianisidine by the Horseradish peroxidase to give a colorimetric readout to the reaction with a change in absorbance at 450 nm visible wavelength.

First, the network has been tested in liquid buffer without vesicle or gel. The 100 μl reaction system comprised 30 mM disodium pyrophosphate (pH 8.5), 0.46 μM (0.0024 units) Glycine/Glyphosate oxidase H244K from Bacillus Subtilis (Biovision #7845), 0.5 mM 0-Dianisidine dihydrochloride, 0.25 units Horseradish peroxidase and Glyphosate at concentrations ranging from 0 to 600 mM. Reaction was followed for 1 hour at 25° C. by registering the absorbance at 450 nm on a spectrophotometer.

In a second example, this first network can comprise:

b) the enzyme GST-Bacillus licheniformis Mut #1 or SCF-4 Glycine/Glyphosate oxidase, the enzyme Horseradish Peroxidase and the Amplex red. In the presence of Glyphosate, 2-amino phosphonate and H₂O₂ are produced by the Glycine/Glyphosate oxidase. Afterward, the H₂O₂ is co-processed with the Amplex red by the Horseradish peroxidase to give a colorimetric (red) and fluorescent readout to the reaction.

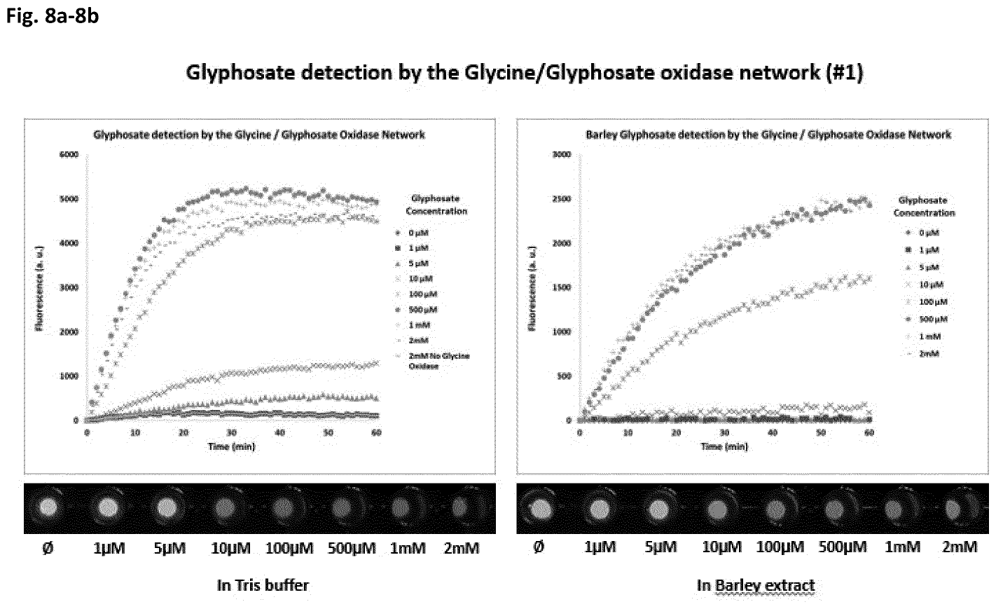

First, the network has been tested in liquid buffer without vesicle or gel (FIGS. 8A-8B). The 100 μl reaction system comprised 50 mM Tris (pH 7.5), 0.46 μM (0.0024 units) Glycine/Glyphosate oxidase GST-BliGO Mut #1 from Bacillus Licheniformis genetically modified and derived from the BliGO-SCF-4 containing 6 single amino-acids mutation compared to the wild type version, 0.2 mM Amplex red, 0.25 units Horseradish peroxidase and Glyphosate at concentrations ranging from 0 to 2 mM. The network has also been tested in the presence of barley extracts (FIG. 8B). Reaction was followed for 1 hour at 25° C. by registering the fluorescence (Excitation at 530 nm/Emission at 590 nm) on a spectrophotometer.

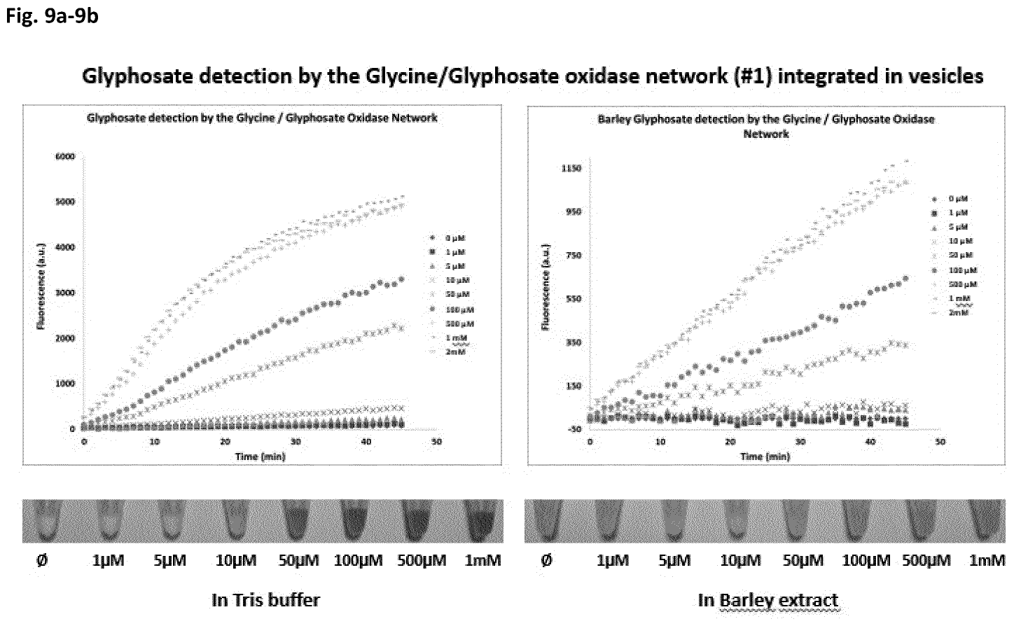

The network has also been tested in vesicle (FIGS. 9A-9B). The vesicles comprised 50 mM Tris (pH 7.5), 0.2 mM Amplex red and 0.25 units Horseradish peroxidase. 0.46 μM (0.0024 units) Glycine/Glyphosate oxidase GST-BliGO Mut #1 from Bacillus licheniformis and Glyphosate at concentrations ranging from 0 to 2 mM were added in the reaction outside of the vesicles. Reaction was followed for 1 hour at 25° C. by registering the fluorescence (Excitation at 530 nm/Emission at 590 nm) on a spectrophotometer.

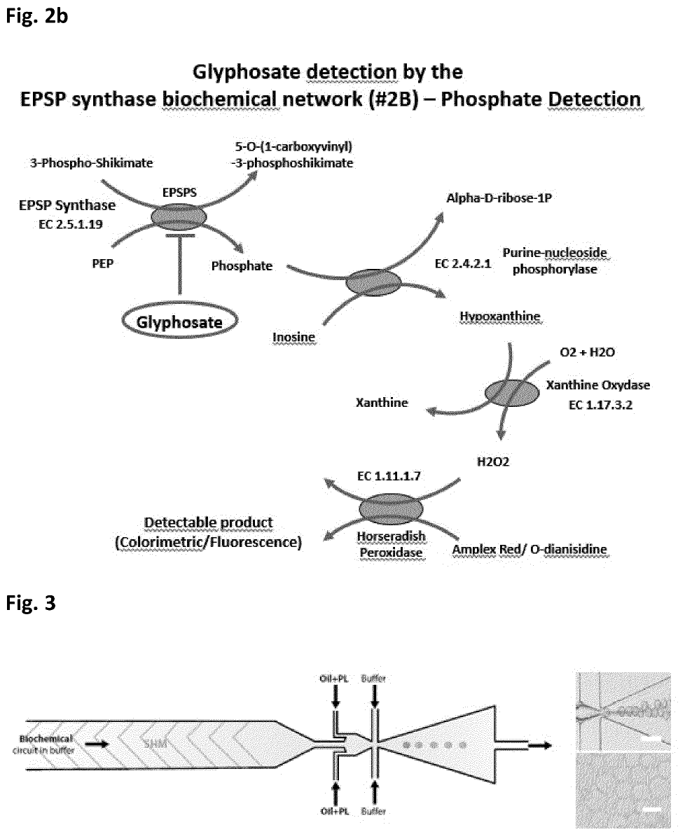

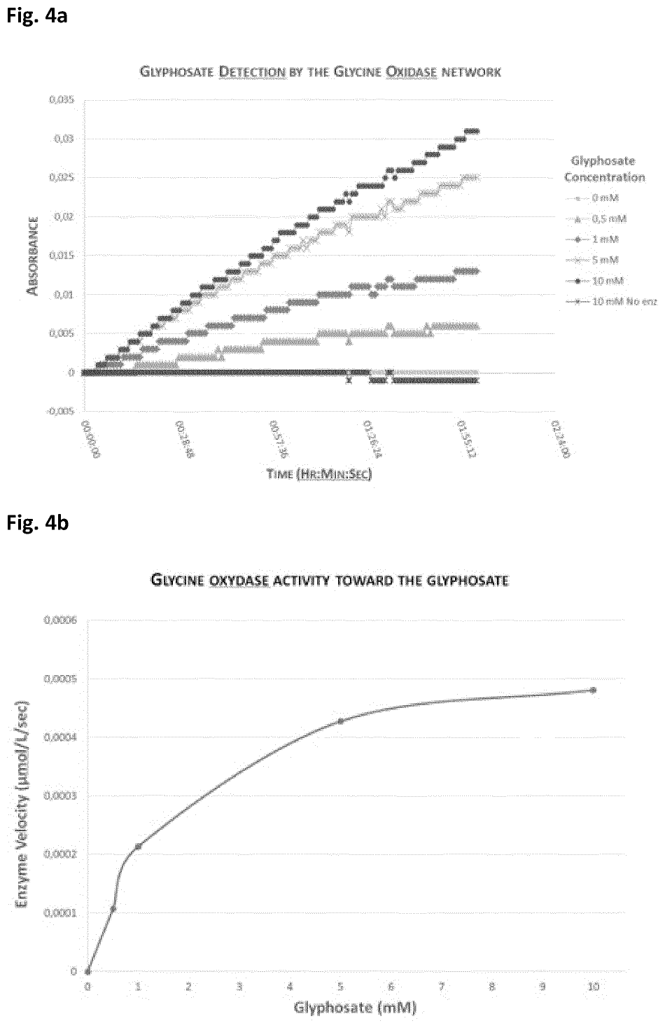

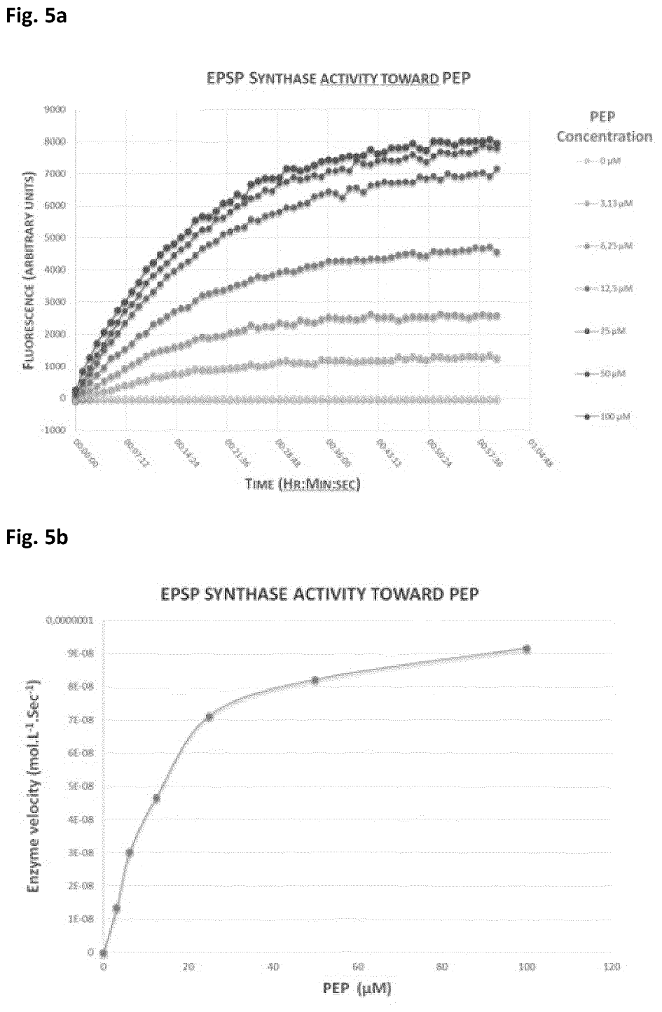

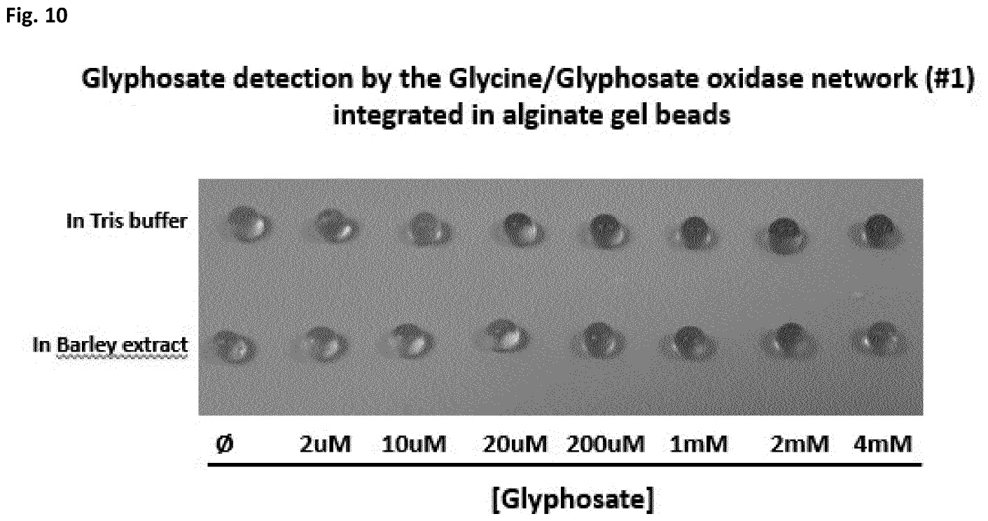

The network has also been tested in alginate beads (FIG. 10). The alginate beads comprised 50 mM Tris (pH 7.5), 0.2 mM Amplex red, 0.25 units Horseradish peroxidase. 0.46 μM (0.0024 units), Glycine/Glyphosate oxidase GST-BliGO from Bacillus Licheniformis on. The beads were dipped in 50 mM Tris buffer pH7,5 or barley extracts containing Glyphosate at concentrations ranging from 0 to 4 mM. Reaction (colorimetry of the beads) was followed for 1 hour at 25° C.
2. The second network combines the activity of 4 enzymes with the first enzyme being the 5-enolpyruvyl Shikimate 3-phosphate-Synthase (EPSP Synthase) (FIGS. 2A-2B).

This network exploits the ability of the Glyphosate to inhibit the activity of the EPSP Synthase. With this network, the level of inhibition depends on the concentration of glyphosate. The entry of the network is composed of: the EPSP synthase that uses the phospho-enol pyruvate (PEP) and the 3-phospho Shikimate to produce 5-O-(1-caroxyvinyl)-3-phosphoshikimate and inorganic phosphate.

Then 2 different networks have been tested:
One that converts the inorganic phosphate (Pi): Pi is combined with Inosine in the presence of the purine nucleoside phosphorylase to give Hypoxanthine. The Hypoxantine lead to Xanthine and H₂O₂ in the presence of Xanthine Oxidase. The Horseradish peroxidase uses the H₂O₂ to convert the O-dianisidine or the Amplex Red and give a colorimetric or fluorimetric signal detectable.

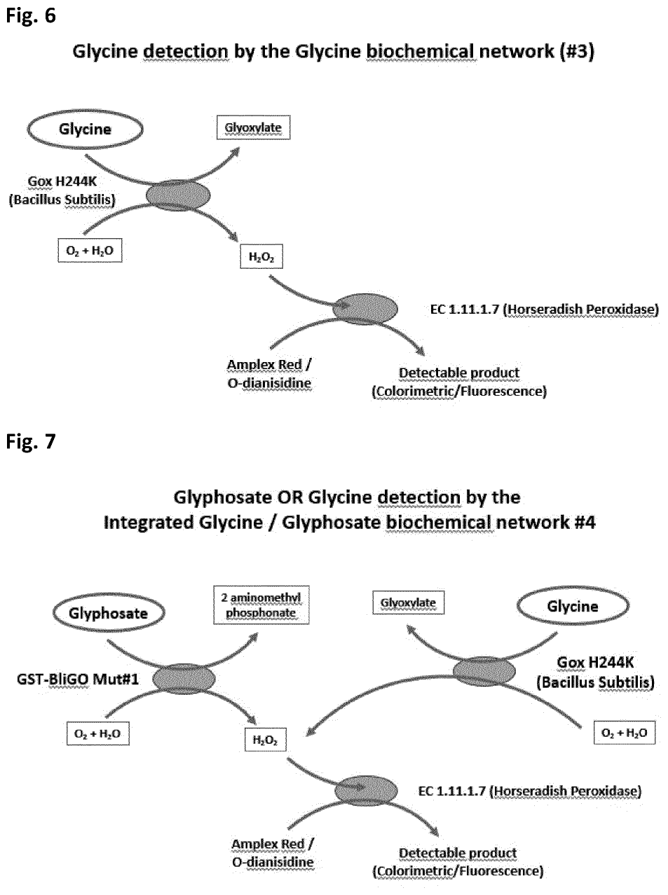

First, the network has been tested in liquid buffer without vesicle or gel. The 100 μl reaction system comprised 50 mM Hepes (pH 7), 50 mM KCl, 0.5 mM Shikimate-3-phosphate, 0.1 unit Xanthine Oxidase, 0.12 μg E. coli EPSP Synthase, 0.2 unit Purine Nucleoside Phosphorylase, 2.25 mM Inosine, 0.5 mM 0-dianisidine dihydrochloride, 0.25 unit Horseradish Peroxidase, Phosphoenol Pyruvate between 0 and 600 μM and Glyphosate at concentrations ranging from 0 to 2 mM. Reaction was followed for 1 hour at 25° C. by registering the absorbance at 450 nm on a spectrophotometer.
One that converts the 5-O-(1-caroxyvinyl)-3-phosphoshikimate produced by the EPSP synthase: 5-O-(1-caroxyvinyl)-3-phosphoshikimate is metabolized by the Chorismate Synthase to give chorismate. This Chorismate is then transformed in pyruvate by the chorismate Lyase. Finaly the pyruvate is used by the Lactate dehydrogenase in the presence of NADH to give lactate and NAD+. The NADH consumption is followed by the change of fluorescence emission at 445 nm (Excitation at 340 nm)—or change of absorbance at 340 nm on a spectrophotometer.
3. The third network (#3) uses the ability of the Bacillus subtilis H244K (Creative enzyme) enzyme Glycine/Glyphosate oxidase to metabolize the Glycine (FIG. 6).

The network comprises: the enzyme Bacillus subtilis H244K Glycine/Glyphosate oxidase, the enzyme Horseradish Peroxidase and the Amplex red. In the presence of Glycine but not Glyphosate, glyoxylate and H₂O₂ are produced by the Glycine/Glyphosate oxidase. Afterward, the H₂O₂ is co-processed with the Amplex red by the Horseradish peroxidase to give a colorimetric (red) and fluorescent readout to the reaction.

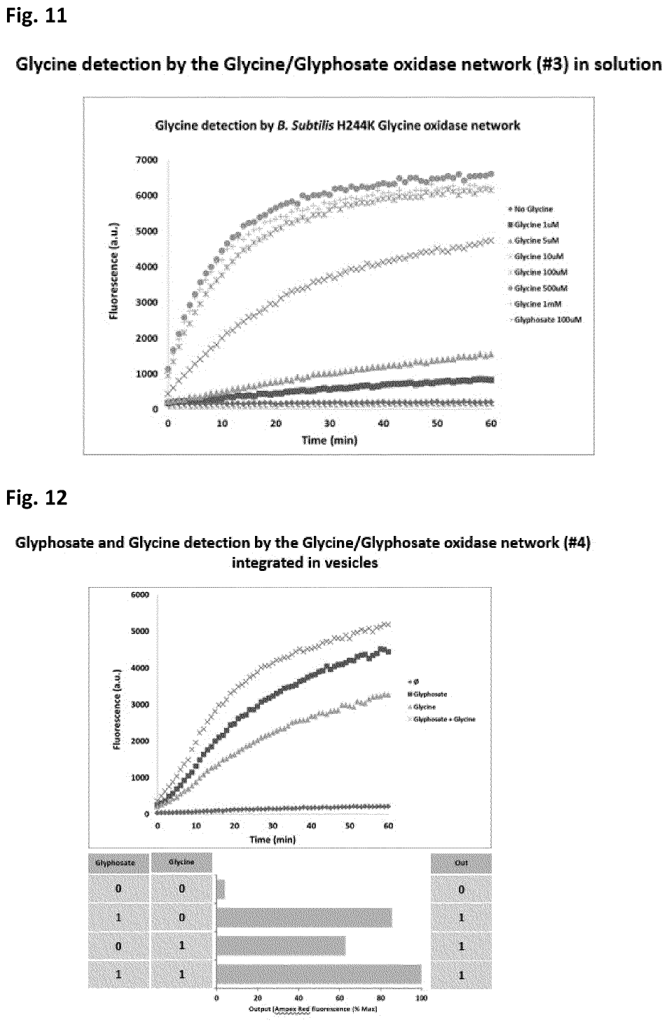

First, the network has been tested in liquid buffer without vesicle or gel (FIG. 11). The 100 μl reaction system comprised 50 mM Tris (pH 7.5), 0.46 μM (0.0024 units) H244K Glycine/Glyphosate oxidase from Bacillus subtilis genetically modified containing 1 single amino-acids mutation H244K (see Accession Number 031616 Biovision) compared to the wild type version (Creative enzyme NATE-1674), 0.2 mM Amplex red, 0.25 units Horseradish peroxidase and Glycine at concentrations ranging from 0 to 1 mM. Reaction was followed for 1 hour at 25° C. by registering the fluorescence (Excitation at 530 nm/Emission at 590 nm) on a spectrophotometer.
4. The fourth network (#4) combines the first and the third networks in order to detect glycine and glyphosate (FIG. 7).

The network comprises: the enzyme GST-Bacillus licheniformis Mut #1 Glycine/Glyphosate oxidase, the Bacillus subtilis H244K Glycine/Glyphosate oxidase, the enzyme Horseradish Peroxidase and the Amplex red. In the presence of Glycine OR Glyphosate, 2-amino phosphonate, glyoxylate and H₂O₂ are produced by the Glycine/Glyphosate oxidase network #4. Afterward, the H₂O₂ is co-processed with the Amplex red by the Horseradish peroxidase to give a colorimetric (red) and fluorescent readout to the reaction.

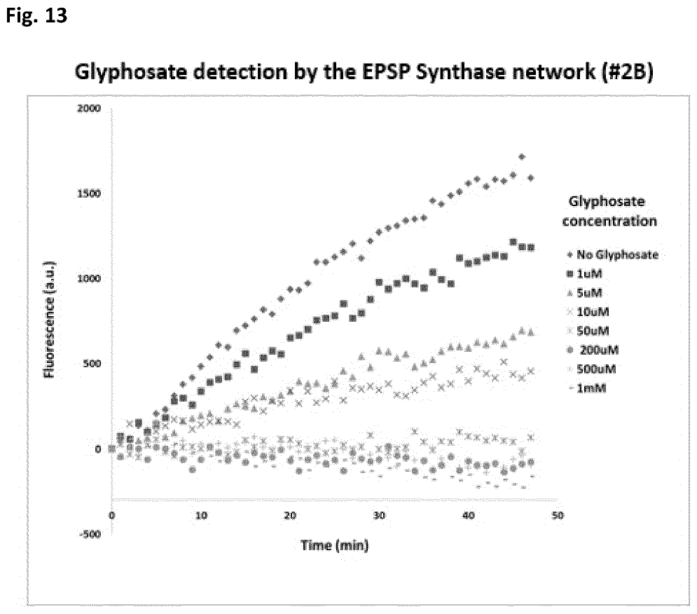

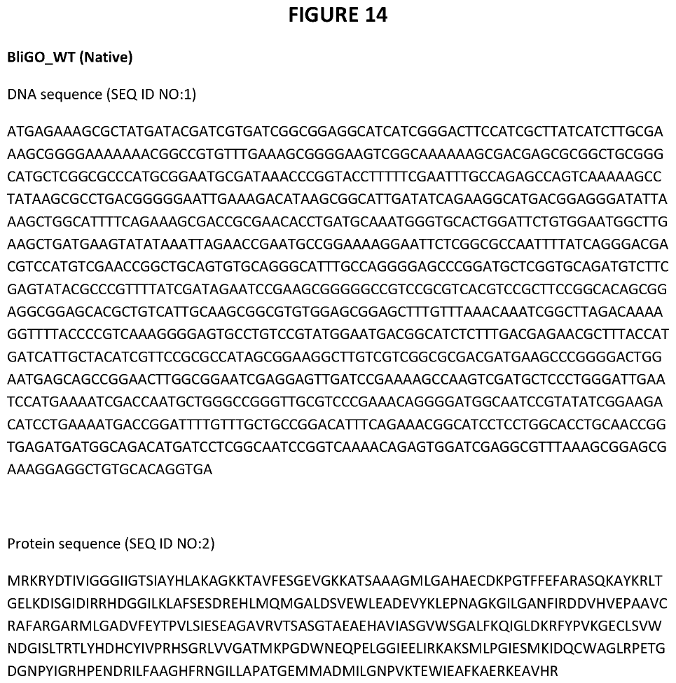

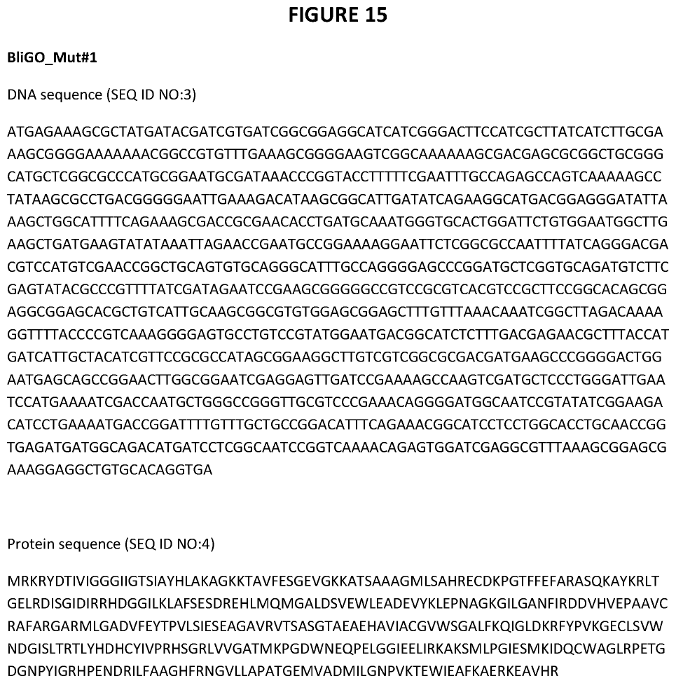

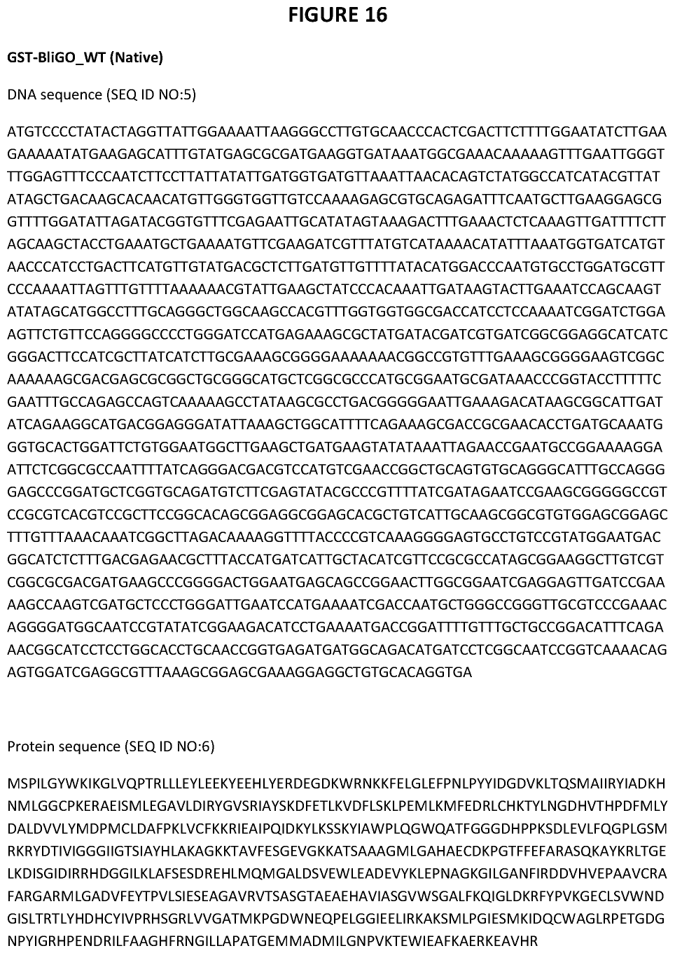

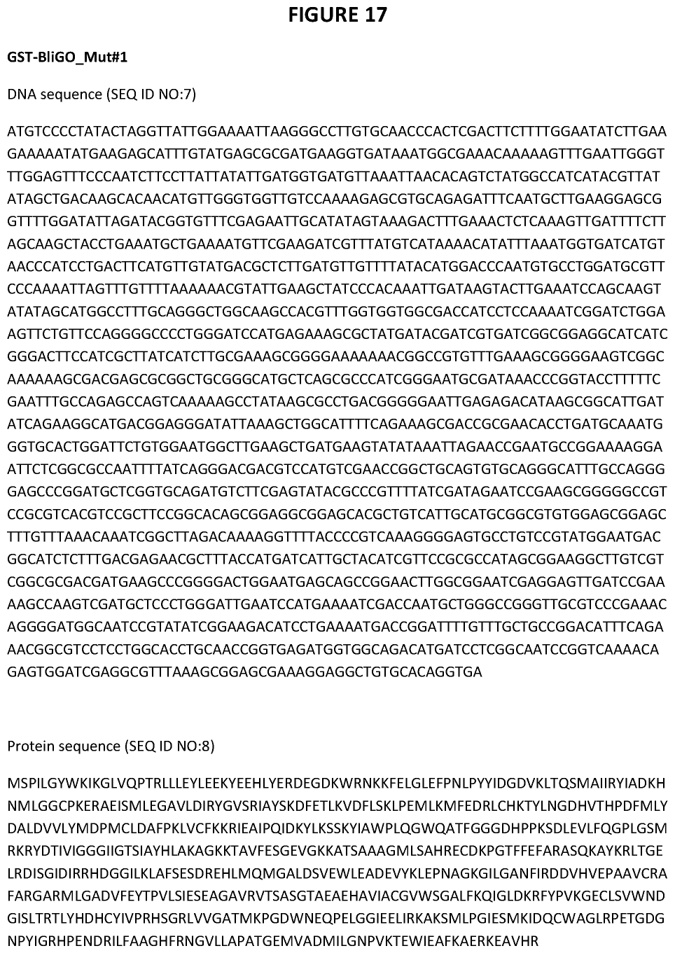

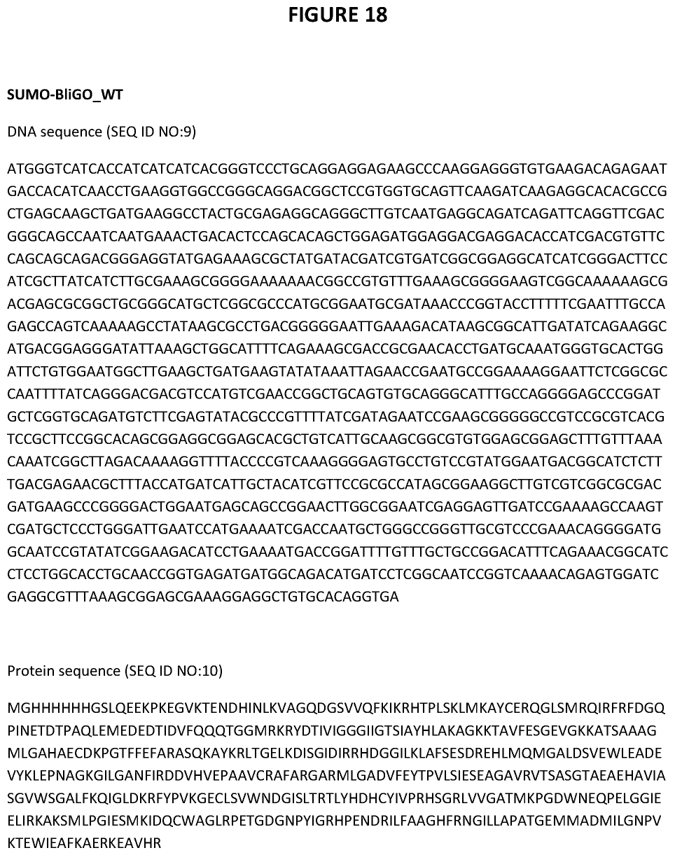

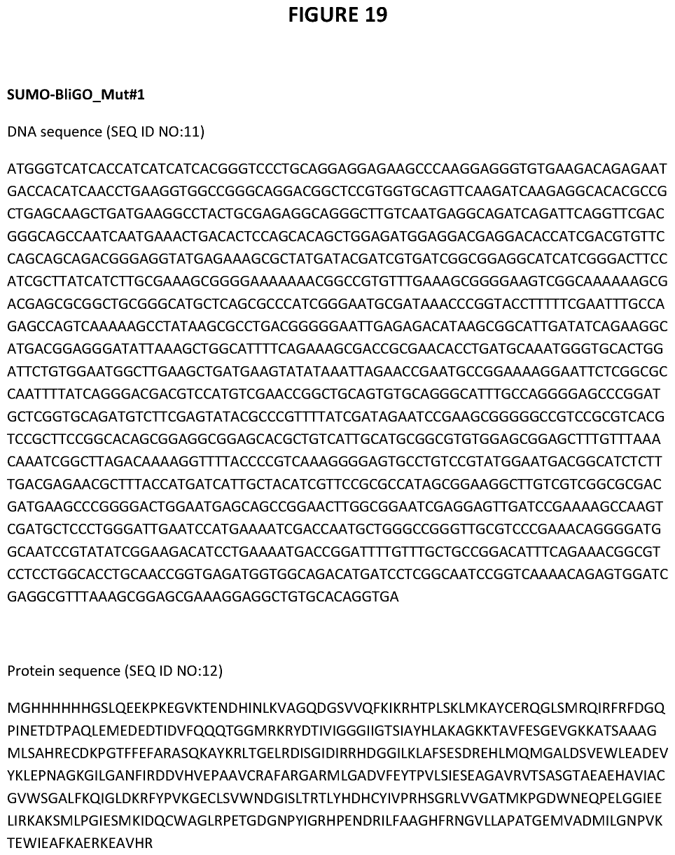

The network has been tested in vesicles (FIG. 12). The vesicles comprised 50 mM Tris (pH 7.5), 0.2 mM Amplex red and 0.25 units Horseradish peroxidase. 0.46 μM (0.0024 units) Glycine/Glyphosate oxidase GST-BliGO Mut #1 from Bacillus Licheniformis, 0.46 μM (0.0024 units) H244K Glycine/Glyphosate oxidase from Bacillus subtilis genetically modified containing 1 single amino-acids mutation H244K compared to the wild type version (Creative enzyme NATE-1674) and Glyphosate at concentrations ranging from 0 to 2 mM were added in the reaction outside of the vesicles. Reaction was followed for 1 hour at 25° C. by registering the fluorescence (Excitation at 530 nm/Emission at 590 nm) on a spectrophotometer.

### Example 2: Setup of the Vesicles to Encapsulate the Biochemical Networks (Refer to Courbet et al. Mol. Sys. Biol. 2018)

We identified a universal and robust macromolecular architecture capable of supporting the modular implementation of in vitro biosensing/biocomputing processes. This architecture is capable of (i) stably encapsulating and protecting arbitrary biochemical circuits irrelevant of their biomolecular composition, (ii) discretizing space through the definition of an insulated interior containing the synthetic circuit, and an exterior consisting of the medium to operate in (e.g. a clinical sample), (iii) allowing signal transduction through selective mass transfer of molecular signals (i.e. biomarker inputs), and (iv) supporting accurate construction through thermodynamically favourable self-assembling mechanisms. The vesicles architecture we propose in this study is made of phospholipid bilayer membranes.

We relied on the development of a method that would simultaneously support (i) membrane unilamellarity, (ii) encapsulation efficiency and stoichiometry, (iii) monodispersity, and (iv) increased stability/resistance to osmotic stress. For this purpose, we developed a custom microfluidic platform and designed PDMS-based microfluidic chips in order to achieve directed self-assembly of a synthetic phospholipid (DPPC) into calibrated, custom-sized membrane bilayers encapsulating low copy number of biochemical species. Briefly, this strategy relied on flowfocusing droplet generation channel geometries that generate waterin-oil-in-water double-emulsion templates (W-O-W: biochemical circuit in PBS—DPPC in oleic acid—aqueous storage buffer with a low concentration of methanol). After double-emulsion templates formation, DPPC phospholipid membranes are precisely directed to self-assemble during a controlled solvent extraction process of the oil phase by methanol present in buffer (FIG. 3). This microfluidic design also integrates a device known as the staggered herringbone mixer (SHM) (Williams et al, 2008) to enable efficient passive and chaotic mixing of multiple upstream channels under Stokes flow regime. We reasoned that laminar concentration gradients could prevent critical mixing of biochemical parts, precise stoichiometry, and efficient encapsulation. We hypothesized that synthetic biochemical circuits immediately homogenized before assembly could standardize the encapsulation mechanism and reduce its dependency on the nature of insulated materials. Moreover, this design allowed for fine-tuning on stoichiometry via control on the input flow rates, which proved practical to test different parameters for straightforward prototyping of protosensors.

We used an ultrafast camera to achieve real-time monitoring and visually inspect the fabrication process, which allowed estimating around ˜1,500 Hz the mean frequency of vesicles generation at these flow rates. A strong dependence of vesicles generation yields on flow rates was found, which we kept at 1/0.4/0.4 μl/min (storage buffer/DPPC in oil/biochemical circuit in PBS, respectively) to achieve best assembly efficiency. We then analysed the size dispersion of vesicles using light transmission, confocal, and environmental scanning electron microscopy. Monodispersed vesicles with average size parameter of 10 μm and an apparent inverse Gaussian distribution were observed. Interestingly, biochemical circuit insulation did not appear to influence the size distribution of vesicles, which supports the decoupling of the insulation process from the complexity of the biochemical content. Moreover, no evolution of sizes was recorded after 3 months, which demonstrated the absence of fusion events between vesicles. In order to assess the capability of vesicles to encapsulate protein species without leakage, which is a prerequisite to achieve rational design of biochemical information processing, we assayed encapsulation stability using confocal microscopy. To this end, an irrelevant protein bearing a fluorescent label was encapsulated within vesicles, and the evolution of internal fluorescence was monitored over the course of 3 months. The internal fluorescence was found to remain stable, which demonstrated no measurable protein leakage through the vesicle membrane in our storage conditions. In addition, using phospholipid bilayer specific dye, DiIC18, which undergoes drastic increase in fluorescence quantum yield when specifically incorporated into bilayers (Gullapalli et al, 2008, Phys Chem Chem Phys; 10(24): 3548-3560), the complete extraction of oleic acid from the double emulsion and a well-structured arrangement of the bilayer could be visualized. We next sought to assess the encapsulation of biological enzymatic parts inside vesicles. We found that we could retrieve the molecular signatures of the enzymes in the interior of vesicles. Taken together, these findings show that this setup proved capable of generating stable, modular vesicles with high efficiency, and user-defined finely tunable content.

### Example 3: Incorporation of the Vesicles Containing the Biochemical Network of Interest in a Gel Matrix

Once the vesicles containing the biochemical network of interest are ready, they are incorporated into the final format which is a set of gel matrix based beads. The size of the gel beads can be adjusted depending of the end user needs (i.e. 5 mm diameter). The gel is composed of 10% polyvinyl-alcohol (PVA) and 1% sodium-alginate. The mix containing all the components of the biochemical network in vesicles is incorporated in a liquid solution of 10% polyvinyl-alcohol (PVA), 1% sodium-alginate. The biochemical network/PVA/Alginate mix is then dropped in a 0.8M Boric acid/0.2M CaCl₂ solution under agitation with a stir bar. After 30 minutes, the beads are rinsed 2 times in water and dropped in a 0.5M sodium sulphate buffer for 90 minutes. The beads are rinsed 2 times in cold PBS and conserved in PBS at 4° C.

### Example 4: Detection of the Glyphosate/Quantification Results

1. Detection of Glyphosate by the Glycine/Glyphosate Oxidase network

1.1 By using the 0-Dianisidine dihydrochloride, we followed the Glyphosate oxidation by the Glycine/Glyphosate Oxidase that is dependent of Glyphosate concentration (FIG. 1, 4A, 4B). The color change of the beads was followed by the change of absorbance at 450 nm on a spectrophotometer. It allowed us to determine an affinity (Km) of the Glycine/Glyphosate Oxidase for the glyphosate that is 2.5 mM and a Vmax of 6×10⁻⁹ mol/L/sec.

1.2 Detection of Glyphosate in Tris buffer and barley extracts by the Glycine/Glyphosate Oxidase network (#1) in liquid, vesicle or gel)

1.2.1 Preparation of Barley extracts for subsequent Glyphosate detection First, Barley grains where grinded and sifted. The powder was ressuspended with Tris 100 mM pH 7,5 and incubated for 30 minutes on a wheel at room temperature. The extract was centrifugated for 10 minutes at 4000 g. The supernatant was filtered with 0.2 micrometer cutoff syringe filter and conserved at 4° C. before analysis.

1.2.2 By using the Amplex red, we followed the Glyphosate oxidation by the Glycine/Glyphosate Oxidase that is dependent of Glyphosate concentration (FIG. 8A-8B). The reaction was followed by the change of fluorescence on a fluorimeter (Excitation at 530 nm/Emission at 590 nm). In parallel we followed the color change of the reaction that is dependent on the glyphosate concentration. It allowed us to detect the glyphosate not only in simple buffered medium (FIG. 8A) but also in complex barley extracts (FIG. 8B).

1.2.3 Detection of Glyphosate by the Glycine/Glyphosate Oxidase network (#1) in vesicles

After incorporating a part of the network in the vesicles (HRP, Amplex Red, Tris 50 mM buffer pH 7,5) and outside the vesicles (Glycine/Glyphosate oxidase), we followed the Glyphosate oxidation that is dependent of Glyphosate concentration (FIG. 9A-9B)). The reaction was followed by the change of fluorescence on a fluorimeter (Excitation at 530 nm/Emission at 590 nm). In parallel we followed the color change of the reaction that is dependent on the glyphosate concentration. It allowed us to detect the glyphosate not only in simple buffered medium (FIG. 9A) but also in complex barley extracts (FIG. 9B)

1.2.4 Detection of Glyphosate by the Glycine/Glyphosate Oxidase network (#1) in gel beads

The full Glycine/Glyphosate Oxidase network was incorporated in alginate gel beads.

We followed the Glyphosate oxidation that is dependent of Glyphosate concentration (FIG. 10). The reaction was followed by the change of beads color (red). Once again, it allowed us to detect the glyphosate not only in simple buffered medium (FIG. 10 (first line)) but also in complex barley extracts (FIG. 10, second line).

2. Detection of Glyphosate by the EPSP Synthase network plugged to phosphate detection

By using the O-Dianisidine dihydrochloride or Amplex red, we followed the Glyphosate inhibition of the EPSP Synthase that is dependent of Glyphosate concentration (FIG. 2B, 5A, 5B, 7). The reaction was followed by the change of fluorescence on a fluorimeter (Excitation at 530 nm/Emission at 590 nm) or by the change of absorbance at 450 nm on a spectrophotometer. It allowed us to determine the activity of the EPSP synthase toward Phosphoenol Pyruvate (PEP). EPSP affinity for PEP is 14 μM and Vmax is at 10.26×10⁻⁹ mol/L/sec. Moreover it allowed us to detect the glyphosate by its inhibitory effect on the EPSP synthase (FIG. 7).

3. Detection of Glyphosate by the EPSP Synthase network plugged to 5-O-(1-caroxyvinyl)-3-phospho shikimate detection

By monitoring the NADH consumption, we followed the Glyphosate inhibition of the EPSP Synthase that is dependent of Glyphosate concentration (FIG. 2A). The NADH consumption was given by the change of fluorescence emission at 445 nm (Excitation at 340 nm)—or change of absorbance at 340 nm on a spectrophotometer.

### Example 5: Detection of the Glycine/Quantification Results

1. Detection of Glycine in Tris buffer by the Glycine/Glyphosate Oxidase network (#3) in liquid (no vesicle/no gel)

By using the Amplex red, we followed the Glycine oxidation by the Glycine/Glyphosate Oxidase (Glycine/Glyphosate oxidase H244K Glycine/Glyphosate oxidase from Bacillus subtilis genetically modified containing 1 single amino-acids mutation H244K compared to the wild type version (Creative enzyme NATE-1674)) that is dependent of Glycine concentration (FIG. 11). The reaction was followed by the change of fluorescence on a fluorimeter (Excitation at 530 nm/Emission at 590 nm). It allowed us to detect the glycine.

### Example 6: Detection of Glyphosate and Glycine/Quantification Results

1. Detection of Glyphosate and glycine in Tris buffer by the Glycine/Glyphosate Oxidase network (#4) in vesicles

In this example we took advantage of the specificity of the GST-BliGO Mut #1 toward glyphosate compared to glycine and of the specificity of the Glycine/Glyphosate oxidase H244K from Bacillus subtilis toward glycine compared to glyphosate. Indeed, the GST-BliGO Mut #1 is derived from the BliGO SCF4 mutant developed by Zhang et al. (2016). This mutant has an 8-fold increase of affinity (1.58 mM) toward glyphosate and its activity to glycine decreased by 113-fold compared to WT. This mutant was developed to increase plants resistance to glyphosate and we used it as a basis for glyphosate biosensing.

After incorporating a part of the network in the vesicles (HRP, Amplex Red, Tris 50 mM buffer pH7,5) and outside the vesicles (Glycine/Glyphosate oxidase H244K Glycine/Glyphosate oxidase from Bacillus subtilis genetically modified containing 1 single amino-acids mutation H244K compared to the wild type version (Creative enzyme NATE-1674) and GST-BliGO Mut #1 from Bacillus Licheniformis derived from the BliGO-SCF-4 genetically modified and containing 6 single amino-acids mutation compared to the wild type version, we followed the Glyphosate AND/OR glycine oxidation that is dependent of Glyphosate and Glycine concentration (FIG. 12)). The reaction was followed by the change of fluorescence on a fluorimeter (Excitation at 530 nm/Emission at 590 nm). It allowed us to detect the glyphosate alone, the glycine alone and the glyphosate and glycine combination. (FIG. 12)

### CONCLUSION AND DISCUSSION

This study demonstrated that the method and the composition according to the present invention are highly promising tools to perform detection and quantification of pesticides or endocrine disruptors, potentially multiplexed. We showed that this technology could be successfully applied to solve real environmental problems and demonstrated that the method and the composition of the present invention could overcome several hurdles faced by classical diagnosis tools in this field.

1. A method to detect the presence or the absence, and/or to quantify the amount of at least one target analyte in a sample, the method comprising the steps of: from a sample containing or susceptible to contain the target analyte;
a) contacting the sample with a composition comprising biochemical elements forming a biochemical network, said biochemical network comprising as biochemical element at least one enzyme having as substrate or as inhibitor or as activator said target analyte which is desired to be detected and/or quantified, and wherein:
at least one of said biochemical elements forming a biochemical network is encapsulated in one micro- or nano-vesicle (named vesicle) permeable or not to the target analyte; or
at least two of said biochemical elements forming a biochemical network are encapsulated in two distinct vesicles permeable or not to the target analyte,
wherein:
i) said at least one target analyte which is desired to be detected or quantify in the sample is the glyphosate;
ii) the biochemical network is capable of:
generating at least one specific readable/measurable output signal only in presence of the target analyte when said target analyte is a substrate of the enzyme of said biochemical network; or
inhibiting the specific readable/measurable output signal generated by said biochemical network only in presence of the target analyte when said target analyte is an inhibitor of the enzyme of said biochemical network, and,
b) determining the rate and/or level of the specific readable/measurable output signal produced by the biochemical network, the rate and/or level obtained being correlated to the presence or the absence and/or the amount of the target analyte in the sample.

2. The method of claim 1, wherein the sample susceptible to contain the target analyte is selected from the group consisting of fluid or solid material sample, preferably environmental material sample, vegetal material, water, beverage, food products, soil extracts, industrial material, food production, plant extract, physiologic fluid or tissue from living organism.

3. The method of claim 1, wherein the presence and/or absence and/or the amount of the target analyte is detected and/or quantified by the measure of a signal selected from the group consisting of: visible colorimetric measurement, fluorescence, luminescence, spectroscopy (i.e. infra-red, Raman), chemical compound or particle (electron) production.

4. The method of claim 1, to detect the presence or the absence, and/or to quantify the amount of at least a second target analyte in a sample wherein said second analyte is either a substrate, inhibitor or activator of the same at least one biochemical network enzyme wherein at least one of said biochemical elements forming a biochemical network is encapsulated in said vesicle.

5. The method of claim 1, to detect the presence or the absence, and/or to quantify the amount of at least a second target analyte in a sample, wherein:
said second analyte is a substrate inhibitor or activator of a second distinct biochemical network enzymes, and one of said biochemical elements forming said second biochemical network is encapsulated in the same or in another distinct vesicle, or set of vesicles; and
said two distinct biochemical networks (interconnected or not) generate a different readable/measurable output signal.

6. The method of claim 1, wherein the second target analyte which is desired to detect and/or to quantify is a pesticide or an endocrine disruptor selected from the group consisting of:
a) pesticide and/or an endocrine disruptor molecule which is a specific substrate of an enzyme activity, activity which can produce in one step, or more, a specific readable/measurable output signal;
b) pesticide and/or an endocrine disruptor molecule which is a specific inhibitor of an enzyme activity, activity which can produce in one step, or more, a specific readable/measurable output signal; and
c) pesticide or an endocrine disruptor molecule which is a specific activator of an enzyme activity, activity which can produce in one step, or more, a specific readable/measurable output signal.

7. The method of claim 6, wherein the second target analyte which is desired to detect and/or to quantify is a pesticide or an endocrine disruptor selected from the group consisting of:
Chlordecone, Neonicotinoid, Organochlorides, Succinate dehydrogenase inhibitor (SDHI), carbamates, dioxine (PCDD), polychlorobiphenyle (PCB), 17-beta oestradiol, 17-alpha ethylene oestradiol, bisphenol (PBDE), phthalates and heavy metal.

8. The method of claim 1, wherein the at least one biochemical network enzyme which is comprised in the composition in step a), encapsulated in said vesicle or not encapsulated, is selected from the group consisting of:
glycine/glyphosate oxidase (EC 1.4.3.19), preferably the native (wild type/WT) glycine/glyphosate oxidase from Bacillus subtilis which can be obtained as recombinant protein, or homolog sequence thereof having at least 70% identity with the WT protein sequence and exhibiting glycine/glyphosate oxidase activity; and
5-enolpyruvylshikimate-3-phosphate (EPSP) synthase (EC 2.5.1.19).

9. The method of claim 1, wherein the at least one biochemical network enzyme which is comprised in the composition in step a), encapsulated in said vesicle or not encapsulated, is the glycine oxidase (GO) from the marine bacteria Bacillus licheniformis ((BliGO) which has been cloned and which shows at least 62% similarity to the standard GO from Bacillus subtilis, or homolog BliGO sequence thereof having at least 70% identity with the BliGO WT protein sequence SEQ ID NO:2 and exhibiting GO activity.

10. The method of claim 1, wherein the at least one biochemical network enzyme which is comprised in the composition in step a), encapsulated in said vesicle or not encapsulated, is the mutated glycine oxidase (GO) from the marine bacteria Bacillus licheniformis genetically modified and containing 6 single amino-acids mutation compared to the wild type version BliGO-WT, named BliGO-SCF-4 or the BliGO-Mut having the amino acids sequence SEQ ID NO:4.

11. The method of claim 1, wherein said glycine/glyphosate oxidase comprising a tag which is fused to the glycine/glyphosate oxidase enzyme, preferably a tag selected from the group consisting of maltose-binding protein (MBP), Chitin Binding Protein (CBP), glutathione S-transferase (GST), thioredoxin (TRX), NUS A, ubiquitin (Ub), and SUMO (small ubiquitin-related modifier) tags, preferably SUMO and GST tags.

12. The method of claim 11, wherein said is a SUMO or a GST tag.

13. The method of claim 8, wherein said glycine/glyphosate oxidase comprising a tag is selected from the group of:
the GST-BliGO (native/WT) having the DNA sequence SEQ ID NO:5 or the amino acids sequence SEQ ID NOG;
the SUMO-BliGO (native/WT) having the DNA sequence SEQ ID NO:9 or the amino acids sequence SEQ ID NO:10;
the GST-BliGO-Mut having the DNA sequence SEQ ID NO:7 or the amino acids sequence SEQ ID NO:8 and the SUMO-BliGO-Mut having the DNA sequence SEQ ID NO:11 or the amino acids sequence SEQ ID NO:12 and
homolog tagged BliGO sequences thereof as defined above wherein the BliGO sequence exhibits at least 70%.

14. The method of claim 8, wherein the target analyte which is desired to detect and/or to quantify is the glyphosate or derivatives thereof which can be detected or quantified with the same biochemical network enzyme as for glyphosate, and wherein the at least one biochemical network enzyme encapsulated or not in a vesicle is 5-enolpyruvylshikimate-3-phosphate (EPSP) synthase (EPSPS) enzyme (EC 2.5.1.19) the composition further comprising 3-phospho-shikimate and phosphoenolpyruvate (PEP).

15. The method of claim 1, wherein the vesicle is selected from the group consisting of unilamellar or multilamellar vesicles, preferred are lipid vesicles, liposomes or self-assembled phospholipids, or vesicles formed from synthetic polymers or copolymers, said vesicles having preferably an average diameter between 0.05 μm to 500 μm, more preferably between 0.1 μm to 100 μm.

16. The method of 1, wherein the vesicles are trapped in a porous polymeric gel, preferably selected from the group consisting of porous polymeric gel, preferably selected from the group consisting of alginate, chitosan, PVP (polyvinylpyrrolidone), PVA (polyvinyl-alcohol), agarose, sephadex, sepharose, sephacryl gel and mixture thereof.

17. A composition to detect the presence or the absence, and/or to quantify the amount of at least one target analyte in a sample said composition comprising biochemical elements forming a biochemical network encapsulated or not in one or in a set of vesicles permeable or not to the target analyte, said biochemical network comprising as biochemical element at least one enzyme selected from the group of:
glycine/glyphosate oxidase (EC 1.4.3.19), preferably the native (wild type/WT) glycine/glyphosate oxidase which can be obtained as recombinant protein, or homolog sequence thereof having at least 70% identity with the WT protein sequence and exhibiting glycine/glyphosate oxidase activity;
5-enolpyruvylshikimate-3-phosphate (EPSP) synthase (EC 2.5.1.19);
the glycine oxidase (GO) from the marine bacteria Bacillus licheniformis ((BliGO) which has been cloned and which shows at least 62% similarity to the standard GO from Bacillus subtilis, or homolog BliGO sequence thereof having at least 70% identity with the BliGO WT protein sequence and exhibiting GO activity;
the mutated glycine oxidase (GO) from the marine bacteria Bacillus licheniformis ((BliGO)_SCF4 genetically modified and containing 6 single amino-acids mutation compared to the wild type version BliGO-WT or the BliGO-Mut;
a tagged glyphosate oxidase enzyme, preferably with a tag selected from the group consisting of maltose-binding protein (MBP), Chitin Binding Protein (CBP), glutathione S-transferase (GST), thioredoxin (TRX), NUS A, ubiquitin (Ub), and SUMO (small ubiquitin-related modifier) tags, preferably SUMO and GST tags; and
the GST-BliGO (native/WT) having the DNA sequence SEQ ID NO:5 or the amino acids sequence SEQ ID NO:6;
the SUMO-BliGO (native/WT) having the DNA sequence SEQ ID NO:9 or the amino acids sequence SEQ ID NO:10;
the GST-BliGO-Mut having the DNA sequence SEQ ID NO:7 or the amino acids sequence SEQ ID NO:8 and the SUMO-BliGO-Mut having the DNA sequence SEQ ID NO:11 or the amino acids sequence SEQ ID NO:12 and
homolog tagged BliGO sequences thereof as defined above wherein the BliGO sequence exhibits at least 70%.

18. A composition wherein the target analyte, the biochemical elements, the biochemical network and the vesicles have the characteristic as defined in claim 1.

19. A kit or a device to detect the presence or the absence, and/or to quantify the amount of at least one target analyte in a sample said kit comprising a container containing the composition of claim 17, trapped in a porous polymeric gel, preferably selected from the group consisting of alginate, chitosan, PVP, PVA, agarose, sephadex, sepharose, sephacryl gel and mixture thereof.
